(19) United States
(12) Patent Application Publication
(10) Pub. No.: US 2004/0180943 A1
(43) Pub. Date: Sep. 16, 2004

(54) Oxazolone analogs as amyloid aggregation inhibitors and for the treatment of alzheimer's disease and disorders related to amyloidosis

(76) Inventors: Corinne Elizabeth Augelli-Szafran, Ann Arbor, MI (US); Annette Theresa Sakkab, Northville, MI (US); Tomoyuki Yasunaga, Ibaraki (JP)

Correspondence Address: WARNER-LAMBERT COMPANY, 2800 PLYMOUTH RD, ANN ARBOR, MI, 48105, US

(21) Appl. No.: 10/624,243

(22) Filed: Jul. 22, 2003

Related U.S. Application Data
(60) Provisional application No. 60/397,901, filed on Jul. 23, 2002.

(51) Int. Cl. A61K 31/421

(52) U.S. Cl. 514/376

(57) ABSTRACT

Disclosed are compounds of the Formula I

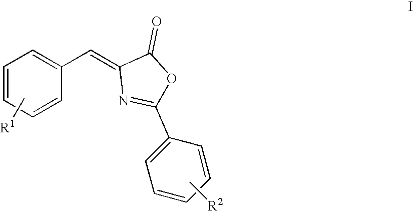

and their use in a method of inhibiting the aggregation of amyloid proteins and in a method of imaging amyloid deposits.

This Application claims the benefit of U.S. provisional application No. 60/397,901 filed on Jul. 23, 2002.

### FIELD OF THE INVENTION

This invention relates to compounds useful for inhibiting amyloid protein aggregation and imaging amyloid deposits. In addition, this invention relates to a method of treating Alzheimer's disease and disorders related to amyloidosis.

### SUMMARY OF THE RELATED ART

Amyloidosis is a condition characterized by the accumulation of various insoluble, fibrillar proteins in the tissues of a patient. The fibrillar proteins that comprise the accumulations or deposits are called amyloid proteins. While the particular proteins or peptides found in the deposits vary, the presence of fibrillar morphology and a large amount of β-sheet secondary structure is common to many types of amyloids. An amyloid deposit is formed by the aggregation of amyloid proteins, followed by the further combination of aggregates and/or amyloid proteins.

The presence of amyloid deposits has been shown in various diseases, each with its particular associated protein, such as Mediterranean fever, Muckle-Wells syndrome, idiopathetic myeloma, amyloid polyneuropathy, amyloid cardiomyopathy, systemic senile amyloidosis, hereditary cerebral hemorrhage with amyloidosis, Alzheimer's disease, Down syndrome, scrapie, Creutzfeldt-Jakob disease, kuru, Gerstmann-Sträussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, sickle cell anemia, Parkinson's disease, and Islets of Langerhans diabetes type 2 insulinoma.

Alzheimer's disease is a degenerative brain disorder characterized clinically by progressive loss of memory, cognition, reasoning, judgement, and emotional stability that gradually leads to mental deterioration and ultimately death. Because Alzheimer's disease and related degenerative brain disorders are a major medical issue for an increasingly aging population, the need for new treatments and methods for diagnosing the disorders are needed.

A simple, noninvasive method for detecting and quantitating amyloid deposits in a patient has been eagerly sought. Presently, detection of amyloid deposits involves histological analysis of biopsy or autopsy materials. Both methods have major drawbacks. For example, an autopsy can only be used for a postmortem diagnosis.

The direct imaging of amyloid deposits in vivo is difficult, as the deposits have many of the same physical properties (i.e., density and water content) as normal tissues. Attempts to image amyloid deposits directly using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules has provided some selectivity on the periphery of tissues, but has provided for poor imaging of tissue interiors.

Thus, it would be useful to have a noninvasive technique for imaging and quantitating amyloid deposits in a patient. In addition, it would be useful to have compounds that inhibit the aggregation of amyloid proteins to form amyloid deposits. An object of this invention is to provide new compounds that are useful to diagnose and treat diseases associated with amyloidosis.

### SUMMARY OF THE INVENTION

The present invention provides compounds that are useful in a method of inhibiting amyloid protein aggregation, the method comprising the administration of an effective amount of the compound to a subject, preferably mammalian, in need thereof.

The present invention is directed to oxazalone derivatives and their use as inhibitors of amyloid protein aggregation. The compounds of the invention are those having the structure of Formula I

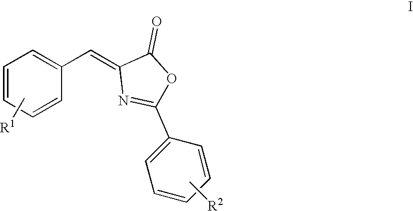

wherein

R¹ is hydrogen, lower alkoxy, hydroxy, aryl, heteroaryl, phenoxy, halogen, hydroxy, cyano, carboxy, alkoxycarbonyl, carbamoyl, sulfamoyl, nitro, trifluoromethyl, amino, mono- or dialkylamino, or

lower alkyl or lower alkenyl unsubstituted or substituted with one, two, or three groups independently selected from oxo, halogen, hydroxy, carboxy, carbamoyl, amino, mono- or dialkylamino, or

aryl or heteroaryl optionally substituted independently with up to three groups selected from halogen, lower alkyl, lower alkoxy, hydroxy, carboxy, alkoxycarbonyl, cyano, nitro, trifluoromethyl, amino, mono- or dialkylamino, carbamoyl, carboxyalkyl, alkoxycarbonylalkyl, sulfamoyl, or carbonylamino;

R² is hydrogen, lower alkyl, lower alkoxy, halogen, hydroxy, aryl, heteroaryl, arylalkyl, heteroarylalkyl, arylalkoxy, heteroarylalkoxy, cyano, carboxy, alkoxycarbonyl, carbamoyl, sulfamoyl, nitro, trifluoromethyl, amino, or mono- or dialkylamino.

The instant invention includes pharmaceutical compositions of compounds of Formula I and a method of treating Alzheimer's disease, the method comprising administering to a patient having Alzheimer's disease a therapeutically effective amount of a compound of Formula I. Also provided is a method for treating disorders related to amyloidosis, the method comprising administering to a patient having disorders related to amyloidosis a therapeutically effective amount of a compound of Formula I.

In a further embodiment, radiolabeled compounds of Formula I are provided, as well as a method for detecting and quantitating amyloid deposits by administering such radiolabeled compound to an animal and measuring the localization thereof in tissues.

Also provided is a method of inhibiting the aggregation of amyloid proteins to form amyloid deposits, the method comprising administering to a patient in need of inhibition of the aggregation of amyloid protein an amyloid protein aggregation inhibiting amount of a compound of Formula I.

In a further embodiment, radiolabeled compounds of Formula I are provided, as well as a method for detecting and quantitating amyloid deposits by administering such radiolabeled compound to an animal and measuring the localization there of in tissues.

### DETAILED DESCRIPTION OF THE INVENTION

The novel compounds encompassed by the instant invention are those described by the general Formula I set forth above, and the pharmaceutically acceptable salts, esters, amides, and prodrugs thereof.

Preferred compounds of Formula I are those in which R¹ is hydrogen, halogen, alkoxy, or heteroaryl; and R² is hydrogen or alkoxy.

Except as expressly defined otherwise, the following definition of terms is employed throughout this specification.

The terms “alkyl,” “lower alkyl,” or “(C₁-C₆)-alkyl” mean a straight or branched hydrocarbon having from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents listed below for aryl.

By “alkoxy,” “lower alkoxy,” or “(C₁-C₆)-alkoxy” in the present invention is meant straight or branched chain alkoxy groups having 1 to 6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

The term “cycloalkyl” means a carbocyclic ring having from 3 to 7 carbon atoms. Examples include cyclopropyl, cyclopentyl, and cycloheptyl. The rings may be substituted with one or more of the substituents listed below for aryl. Examples include 2-aminocyclobutyl.

The term “halogen” includes chlorine, fluorine, bromine, and iodine, and their monovalent radicals.

The term “aryl” means an aromatic carbocyclic group having a single ring (e.g., phenyl), multiple rings (e.g., biphenyl), or multiple condensed rings in which at least one is aromatic (e.g., 1,2,3,4-tetrahydronaphthyl, naphthyl, anthryl, or phenanthryl), unsubstituted or substituted by 1 to 3 substituents selected from alkyl, O-alkyl and S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —CH₂)ₘPO₃H₂, —(CH₂)ₘPO₃(alkyl)₂, —(CH₂)ₘSO₂NH₂, and —(CH₂)ₘSO₂NH-alkyl wherein alkyl is defined as above and m is 0, 1, 2, or 3. A preferable aryl group of the present invention is phenyl. Typical substituted phenyl groups include 2-chlorophenyl, 3-methoxyphenyl, 4-aminophenyl, 3,5-dinitrophenyl, 2,6-dibromo-4-ethoxyphenyl, and 2-hydroxy-3-cyano-5-trifluoromethylphenyl.

The term “aralkyl” or “arylalkyl” means an alkyl moiety (as defined above) substituted with an aryl moiety (also as defined above).

By heteroaryl (aromatic heterocycle) in the present invention is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, benzoxazolyl, and benzodioxolyl. The heterocycle is unsubstituted or substituted by 1 to 3 substituents selected from alkyl, O-alkyl and S-alkyl, OH, SH, —CN, halogen, 1,3-dioxolanyl, CF₃, NO₂, NH₂, NHCH₃, N(CH₃)₂, NHCO-alkyl, —(CH₂)ₘCO₂H, —(CH₂)ₘCO₂-alkyl, —(CH₂)ₘSO₃H, —NH alkyl, —N(alkyl)₂, —CH₂)ₘPO₃H₂, —(CH₂)ₘPO₃(alkyl)₂, —(CH₂)ₘSO₂NH₂ and —(CH₂)ₘSO₂NH-alkyl wherein alkyl is defined as above and m is 0, 1, 2, or 3. A preferable heteroaryl group of the present invention is 2-, 3- or 4-pyridine. Examples of substituted heteroaryl groups include 2-chloropyridin-4-yl, 6-methoxynaphthyridin-2-yl, 6-trifluoromethylpyrimidin-2-yl, 5,6-diethoxybenzimidazol-2-yl, and 4-chloro-5-nitro-7-acetamidobenzoxazol-2-yl.

The symbol “—” means a covalent bond.

The term “pharmaceutically acceptable salt, ester, amide, and prodrug” as used herein refers to those carboxylate salts, amino acid addition salts, esters, amides, and prodrugs of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laureate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as, nontoxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci., 1977;66:1-19 which is incorporated herein by reference.)

Examples of pharmaceutically acceptable, nontoxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods.

Examples of pharmaceutically acceptable, nontoxic amides of the compounds of this invention include amides derived from ammonia, primary C₁-C₆ alkyl amines and secondary C₁-C₆ dialkyl amines wherein the alkyl groups are straight or branched chain. In the case of secondary amines, the amine may also be in the form of a 5- or 6-membered heterocycle containing one nitrogen atom. Amides derived from ammonia, C₁-C₃ alkyl primary amides and C₁-C₂ dialkyl secondary amides are preferred. Amides of the compounds of the invention may be prepared according to conventional methods.

The term “prodrug” refers to compounds that are rapidly transformed in vivo to yield the parent compound of the above formulas, for example, by hydrolysis in blood. A thorough discussion is provided in Higuchi T. and Stella V., Pro-drugs as Novel Delivery Systems, Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

All of the foregoing pharmaceutically acceptable salts, esters, amides, and prodrugs are readily prepared and used by pharmaceutical scientists and medical personnel to whom this invention is directed. Such compounds are those that are pharmaceutically acceptable and can be administered to animals, including humans, for the treatments and other uses described herein.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Certain of the compounds of the present invention possess one or more chiral centers and each center may exist in the R or S configuration. The present invention includes all diastereomeric, enantiomeric, and epimeric forms as well as the appropriate mixtures thereof. Additionally, the compounds of the present invention may exist as geometric isomers. The present invention includes all cis, trans, syn, anti, entgegen (E), and zusammen (Z) isomers as well as the appropriate mixtures thereof.

Representative compounds of the invention are shown below in Table 1.

TABLE 1

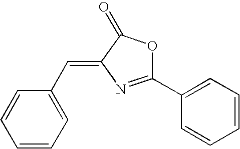

4-Benzylidene-2-phenyl-4H-oxazol-
5-one

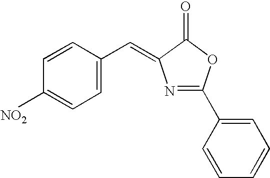

4-(4-Nitro-benzylidene)-2-phenyl-4H-
oxazol-5-one

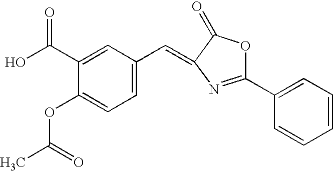

2-Acetoxy-5-(5-oxo-2-phenyl-oxazol-4-
ylidenemethyl)-benzoic acid

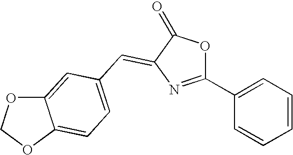

4-1,3-Benzodioxol-5-ylmethylene-2-
phenyl-4H-oxazol-5-one

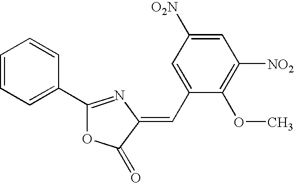

4-(2-Methoxy-3,5-dinitro-benzylidene)-
2-phenyl-4H-oxazol-5-one

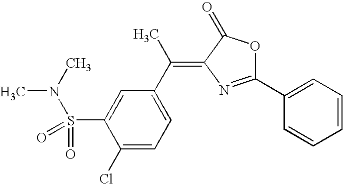

2-Chloro-N,N-dimethyl-5-[1-(5-oxo-
2-phenyl-oxazol-4-ylidene)-ethyl]-
benzenesulfonamide

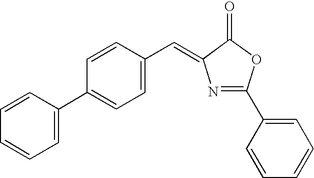

4-Biphenyl-4-ylmethylene-2-phenyl-
4H-oxazol-5-one

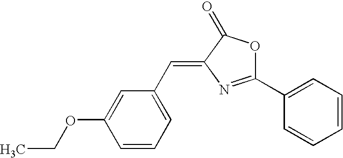

4-(3-Ethoxy-benzylidene)-2-phenyl-
4H-oxazol-5-one

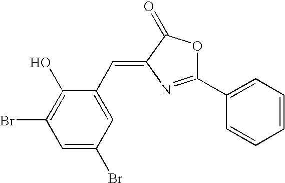

4-(3,5-Dibromo-2-hydroxy-
benzylidene)-2-phenyl-4H-oxazol-5-one

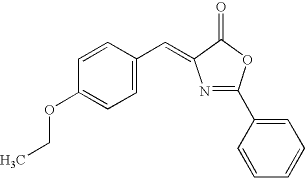

4-(4-Ethoxy-benzylidene)-2-phenyl-
4H-oxazol-5-one

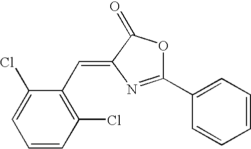

4-(2,6-Dichloro-benzylidene)-2-phenyl-
4H-oxazol-5-one

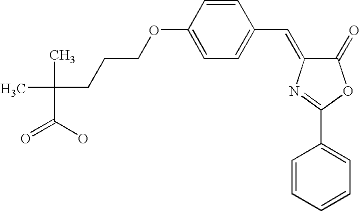

2,2-Dimethyl-5-[4-(5-oxo-2-phenyl-
oxazol-4-ylidenemethyl)-phenoxyl]-
pentanoic acid

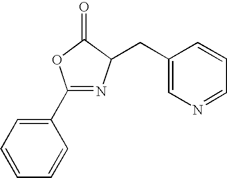

2-Phenyl-4-pyridin-3-ylmethylene-4H-
oxazol-5-one

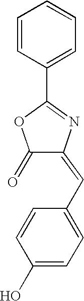

4-(4-Hydroxy-benzylidene)-2-phenyl-
4H-oxazol-5-one

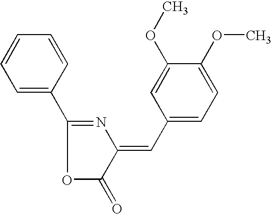

4-(3,4-Dimethoxy-benzylidene)-
2-phenyl-4H-oxazol-5-one

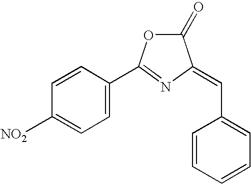

4-Benzylidene-2-(4-nitro-phenyl)-4H-
oxazol-5-one

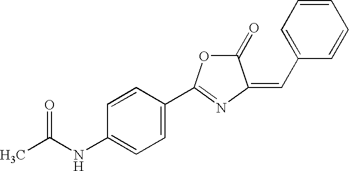

N-[4-(4-Benzylidene-5-oxo-4,5-
dihydro-oxazol-2-yl)-phenyl]-acetamide

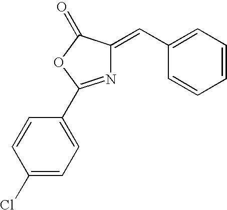

4-Benzylidene-2-(4-chloro-phenyl)-
4H-oxazol-5-one

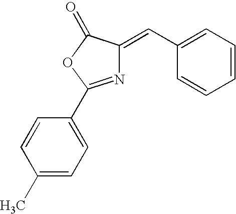

4-Benzylidene-2-p-tolyl-4H-oxazol-
5-one

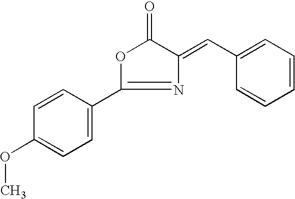

4-Benzylidene-2-(4-methoxy-phenyl)-
4H-oxazol-5-one

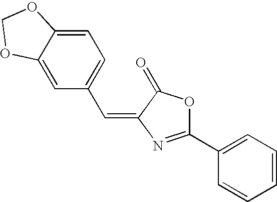

4-Benzo[1,3]dioxol-5-ylmethylene-2-
phenyl-4H-oxazol-5-one

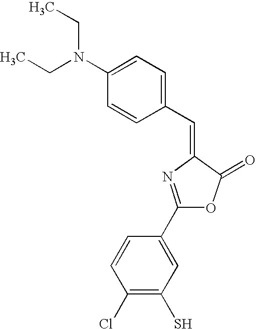

2-(4-Chloro-3-nitro-phenyl)-4-(4-
diethylamino-benzylidene)-4H-
oxazol-5-one

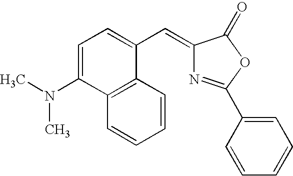

4-(4-Dimethylamino-naphthalen-1-
ylmethylene)-2-phenyl-4H-oxazol-5-one

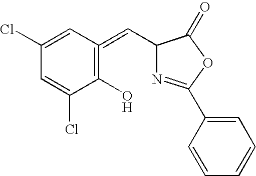

4-(3,5-Dichloro-2-hydroxy-
benzylidene)-2-phenyl-4H-oxazol-5-one

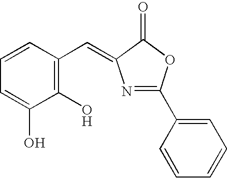

4-(2,3-Dihydroxy-benzylidene)-2-
phenyl-4H-oxazol-5-one

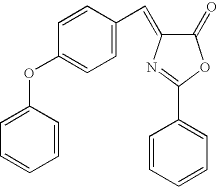

4-(4-Phenoxy-benzylidene)-2-phenyl-
4H-oxazol-5-one

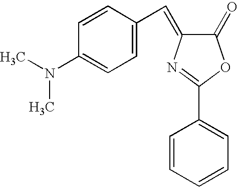

4-(4-Dimethylamino-benzylidene)-2-
phenyl-4H-oxazol-5-one

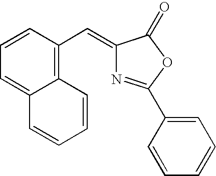

4-Naphthalen-1-ylmethylene-2-
phenyl-4H-oxazol-5-one

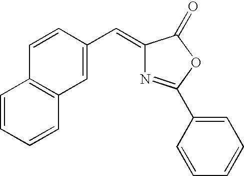

4-Naphthalen-2-ylmethylene-2-phenyl-
4H-oxazol-5-one

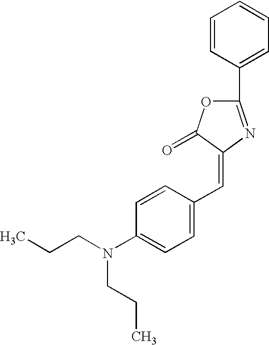

4-(4-Dipropylamino-benzylidene)-2-
phenyl-4H-oxazol-5-one

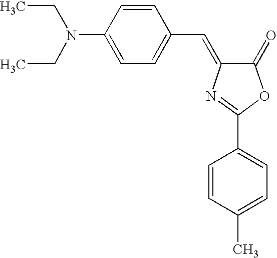

4-(4-Diethylamino-benzylidene)-2-p-
tolyl-4H-oxazol-5-one

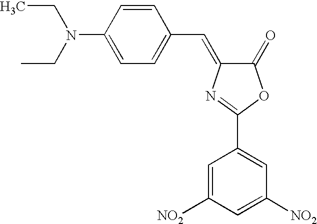

4-(4-Diethylamino-benzylidene)-2-
(3,5-dinitro-phenyl)-4H-oxazol-5-one

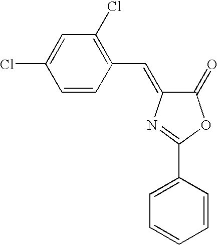

4-(2,4-Dichloro-benzylidene)-2-phenyl-
4H-oxazol-5-one

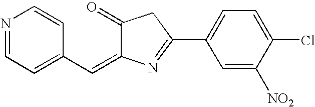

2-(4-Chloro-3-nitro-phenyl)-4-
pyridin-4-ylmethylene-4H-oxazol-5-

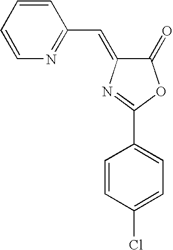

2-(4-Chloro-phenyl)-4-pyridin-2-
ylmethylene-4H-oxazol-5-one

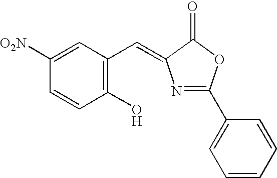

4-(2-Hydroxy-5-nitro-benzylidene)-2-
phenyl-4H-oxazol-5-one

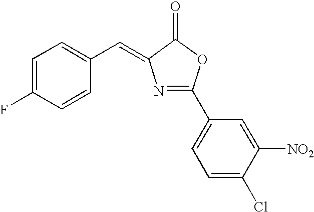

2-(4-Chloro-3-nitro-phenyl)-4-(4-fluoro-
benzylidene)-4H-oxazol-5-one

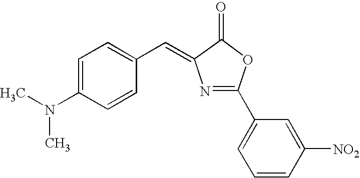

4-(4-Dimethylamino-benzylidene)-2-
(3-nitro-phenyl)-4H-oxazol-5-one

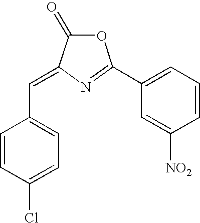

4-(4-Chloro-benzylidene)-2-(3-nitro-
phenyl)-4H-oxazol-5-one

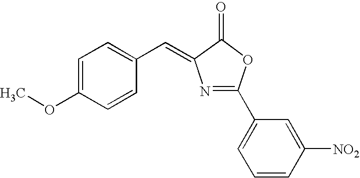

4-(4-Methoxy-benzylidene)-2-(3-
nitro-phenyl)-4H-oxazol-5-one

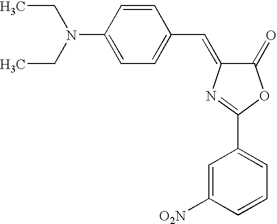

4-(4-Diethylamino-benzylidene)-2-(3-
nitro-phenyl)-4H-oxazol-5-one

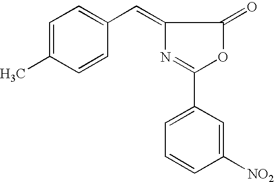

4-(4-Methyl-benzylidene)-2-(3-nitro-
phenyl)-4H-oxazol-5-one

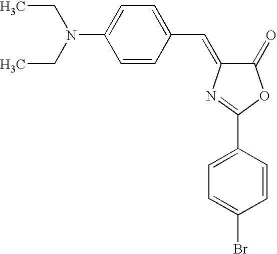

2-(4-Bromo-phenyl)-4-(4-diethylamino-
benzylidene)-4H-oxazol-5-one

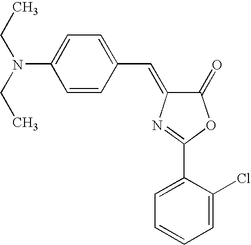

2-(2-Chloro-phenyl)-4-(4-
diethylamino-benzylidene)-4H-
oxazol-5-one

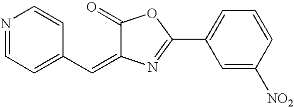

2-(3-Nitro-phenyl)-4-pyridin-4-
ylmethylene-4H-oxazol-5-one

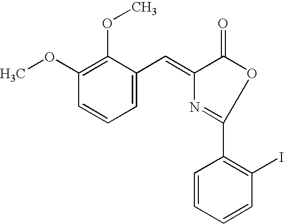

4-(2,3-Dimethoxy-benzylidene)-2-(2-
iodo-phenyl)-4H-oxazol-5-one

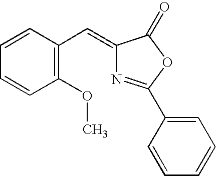

4-2-Methoxy-benzylidene)-2-phenyl-
4H-oxazol-5-one

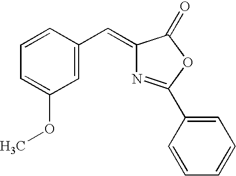

4-(3-Methoxy-benzylidene)-2-phenyl-
4H-oxazol-5-one

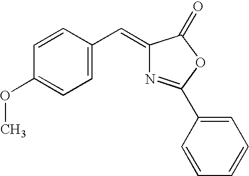

4-(4-Methoxy-benzylidene)-2-phenyl-
4H-oxazol-5-one

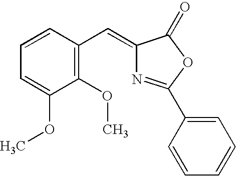

4-(2,3-Dimethoxy-benzylidene)-2-
phenyl-4H-oxazol-5-one

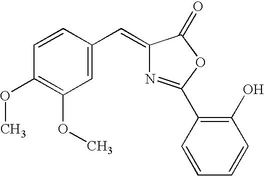

4-(3,4-Dimethoxy-benzylidene)-2-(2-
hydroxy-phenyl)-4H-oxazol-5-one

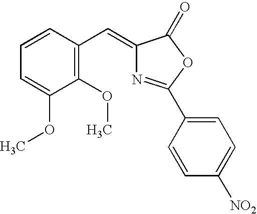

4-(2,3-Dimethoxy-benzylidene)-2-(4-
nitro-phenyl)-4H-oxazol-5-one

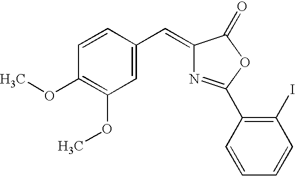

4-(3,4-Dimethoxy-benzylidene)-2-(2-
iodo-phenyl)-4H-oxazol-5-one

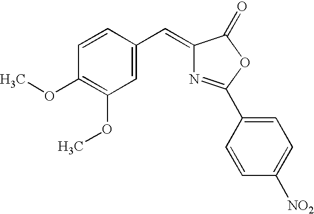

4-(3,4-Dimethoxy-benzylidene)-2-(4-
nitro-phenyl)-4H-oxazol-5-one

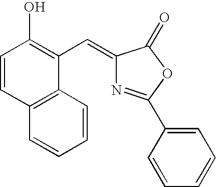

4-(2-Hydroxy-naphthalen-1-
ylmethylene)-2-phenyl-4H-oxazol-5-one

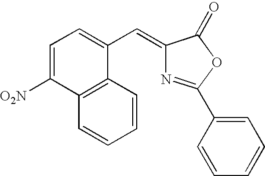

4-(4-Nitro-naphthalen-1-
ylmethylene)-2-phenyl-4H-oxazol-5-

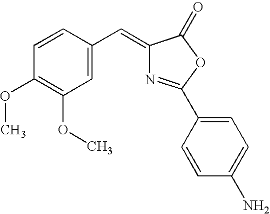

2-(4-Amino-phenyl)-4-(3,4-dimethoxy-
benzylidene)-4H-oxazol-5-one

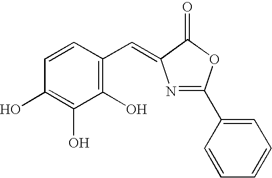

2-Phenyl-4-(2,3,4-trihydroxy-
benzylidene)-4H-oxazol-5-one

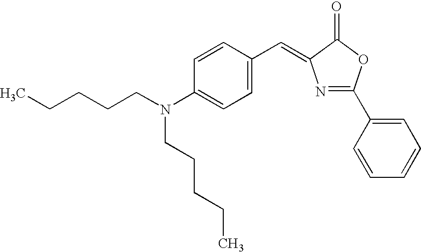

4-(4-Diphenylamino-benzylidene)-2-
phenyl-4H-oxazol-5-one

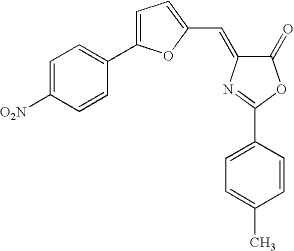

4-[5-(4-Nitro-phenyl)-furan-2-
ylmethylene]-2-p-tolyl-4H-oxazol-5-one

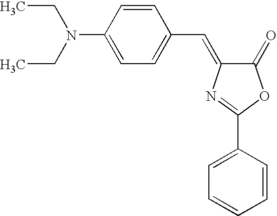

4-(4-Diethylamino-benzylidene)-2-
phenyl-4H-oxazol-5-one

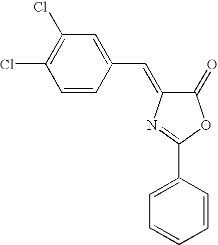

4-(3,4-Dichloro-benzylidene)-2-
phenyl-4H-oxazol-5-one

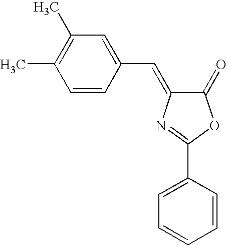

4-(3,4-Dimethyl-benzylidene)-2-phenyl-
4H-oxazol-5-one

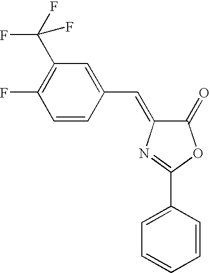

4-(4-Fluoro-3-trifluoromethyl-
benzylidene)-2-phenyl-4H-oxazol-5-one

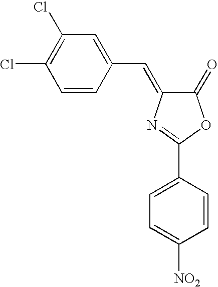

4-(3,4-Dichloro-benzylidene)-2-(4-nitro-
phenyl)-4H-oxazol-5-one

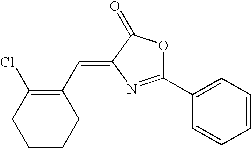

4-(2-Chloro-cyclohex-1-
enylmethylene)-2-phenyl-4H-oxazol-5-one

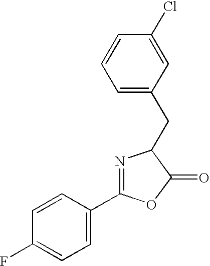

4-(3-Chloro-benzylidene)-2-(4-fluoro-
phenyl)-4H-oxazol-5-one

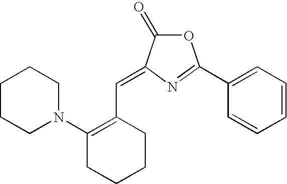

2-Phenyl-4-(2-piperdin-1-yl-
cyclohex-1-enylmethylene)-4H-
oxazol-5-one

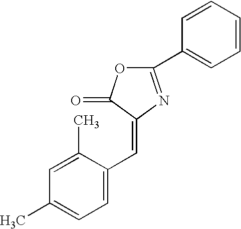

4-(2,4-Dimethyl-benzylidene)-2-phenyl-
4H-oxazol-5-one

TABLE 2

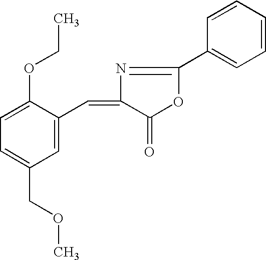

4-(2-Ethoxy-5-methoxymethyl-
benzylidene)-2-phenyl-4H-oxazol-5-one

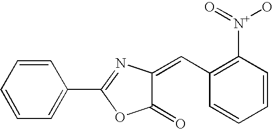

4-(2-Nitro-benzylidene)-2-phenyl-4H-
oxazol-5-one

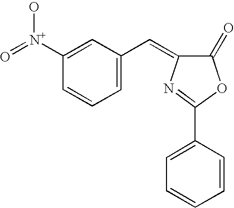

4-(3-Nitro-benzylidene)-2-phenyl-4H-
oxazol-5-one

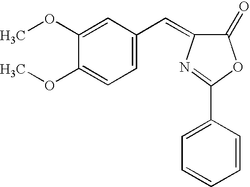

4-(3,4-Dimethoxy-benzylidene)-2-
phenyl-4H-oxazol-5-one

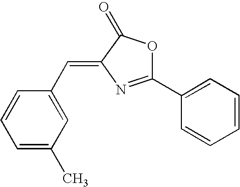

4-(3-Methyl-benzylidene)-2-phenyl-4H-
oxazol-5-one

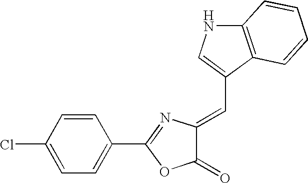

2-(4-Chloro-phenyl)-4-(1H-indol-3-
ylmethylene)-4H-oxazol-5-one

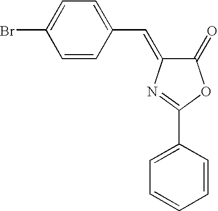

4-(4-Bromo-benzylidene)-2-phenyl-4H-
oxazol-5-one

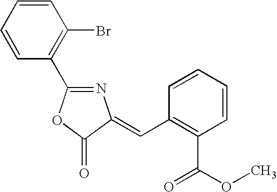

2-[2-(2-Bromo-phenyl)-5-oxo-oxazol
4-ylidenemethyl]-benzoic acid methyl ester

Representative compounds of the present invention, which are encompassed by Formula I, include, but are not limited to the compounds in Table 1 and their pharmaceutically acceptable acid or base addition salts, the esters, amides, or prodrugs thereof.

The invention compounds of Formula I are readily prepared from commercially available reactants, utilizing synthetic methodologies well-known and routinely used by those skilled in the art of synthetic organic chemistry. While the invention compounds can be prepared by any number of alternative processes, typical synthetic routes utilized to prepare illustrative invention compounds are presented in Scheme 1. In Scheme 1, R¹ and R² have the meanings defined above for Formula I.

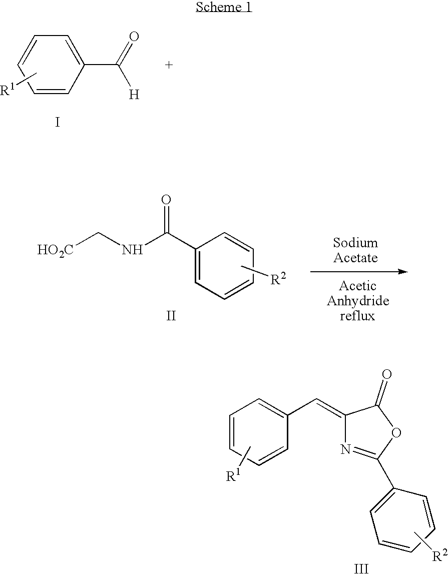

Oxazolone derivatives (III) are synthesized by condensing the corresponding substituted aldehydes (I) (1 eq.) and hippuric acid derivatives (II) (1.01 eq.) in the presence of sodium acetate, acetic anhydride, and refluxing the reaction mixture for 3 hours. Upon cooling the reaction mixture, crude product is precipitated from the reaction mixture by adding ethanol. Purification by chromatography yields desired oxazolones.

The invention also includes radiolabeled compounds of Formula I that are useful for detecting and quantitating amyloid protein deposits. Such radiolabeled compounds are synthesized by standard methods, for example, by using a radiolabeled starting material in any of the foregoing schemes. Typical starting materials are those having a ¹³C, ¹⁹F, ¹⁵N, ¹¹C, or other radioactive atom as part of the molecule.

In the first step of the present method of imaging amyloid deposits, a labeled compound of Formula I is introduced into a tissue or a patient in a detectable quantity. The compound is typically part of a pharmaceutical composition and is administered to the tissue or the patient by methods well-known to those skilled in the art.

Those skilled in the art are familiar with the various ways to detect labeled compounds. For example, MRI, positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

Another example of a suitable label in a compound of Formula I is an atom such as ¹³C, ¹⁵N, or ¹⁹F which can be detected using MRI, which is also sometimes called nuclear magnetic resonance (NMR). In addition, the labeled compounds of Formula I may also be detected by MRI using paramagnetic contrast agents.

Another example of detection is electron paramagnetic resonance (EPR). In this case, EPR probes which are well-known in the art, such as nitroxides, can be used.

The imaging of amyloid deposits can also be carried out quantitatively so that the amount of amyloid deposits can be determined.

In the methods of treating disorders related to amyloidosis according to the present invention, disorders such as Alzheimer's disease, a compound of Formula I can be administered either orally, rectally, parenterally (intravenously, intramuscularly, or subcutaneously), intracisternally, intravaginally, intraperitoneally, intravesically, locally (powders, ointments, or drops), or as a buccal or nasal spray. The invention provides pharmaceutical compositions comprising a compound of Formula I mixed with a carrier, diluent, or excipient.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate, or (a) fillers or extenders, as for example, starches, lactose, sucrose, glucose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft- and hard-filled gelatin capsules using such excipients as lactose or milk sugar, as well as high molecular weight polyethyleneglycols, and the like.

Solid dosage forms such as tablets, dragees, capsules, pills, and granules can be prepared with coatings and shells, such as enteric coatings and others well-known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, and sesame oil, glycerol, tetrahydrofurfuryl alcohol, polyethyleneglycols, and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administrations are preferably suppositories which can be prepared by mixing the compounds of the present invention with suitable nonirritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

In a preferred embodiment of the invention, the compound is labeled and introduced into a patient in a detectable quantity and after sufficient time has passed for the compound to become associated with amyloid deposits, the labeled compound is detected noninvasively inside the patient. In another embodiment of the invention, a labeled compound of Formula I is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I is introduced into the tissue sample. After a sufficient amount of time for the compound to become bound to amyloid deposits, the compound is detected.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals such as horses, dogs, cats, and sheep. Those skilled in the art are also familiar with determining the amount of time sufficient for a compound to become associated with amyloid deposits. The amount of time necessary can easily be determined by introducing a detectable amount of a labeled compound of Formula I into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

The present invention also provides a method of inhibiting the aggregation of amyloid proteins to form amyloid deposits, by administering to a patient in need of inhibition of the aggregation of amyloid protein an amyloid protein inhibiting amount of a compound of Formula I. Those skilled in the art are readily able to determine an amyloid inhibiting amount by simply administering a compound of Formula I to a patient in increasing amounts until the growth of amyloid deposits is decreased or stopped. The rate of growth can be assessed using imaging or by taking a tissue sample from a patient and observing the amyloid deposits therein.

A patient in need of inhibition of the aggregation of amyloid proteins is a patient having a disease or condition in which amyloid proteins aggregate. Examples of such diseases and conditions include Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Alzheimer's disease, Downs syndrome, sickle cell anemia, scrapie, Parkinson's disease, Creutzfeldt-Jakob disease, kuru, Gerstmann-Sträussler-Scheinker syndrome, medullary carcinoma of the thyroid, isolated atrial amyloid, β₂-microglobulin amyloid in dialysis patients, inclusion body myositis, β₂-amyloid deposits in muscle wasting disease, and Islets of Langerhans diabetes type 2 insulinoma.

The compounds of the present invention can be administered to a patient at dosage levels in the range of about 0.1 mg/day to about 1000 mg/day. For a normal human adult having a body weight of about 70 kg, a dosage in the range of about 0.01 mg/kg to about 100 mg/kg of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well-known to those skilled in the art.

Armed with the disclosure provided herein (particularly the schemes and the synthetic examples described above) and knowledge common to all who practice in the field, those of ordinary skill in the art will be able to make and use the entire scope of compounds disclosed herein.

The invention is illustrated further by the following detailed examples which are not to be construed as limiting the invention in scope or spirit to the specific procedures described in them.

The starting materials and various intermediates may be obtained from commercial sources, prepared from commercially available organic compounds, or prepared using well-known synthetic methods.

Representative examples of methods for preparing intermediates of the invention are set forth in the examples below.

As noted above, the compounds of Formula I are useful because of their ability to inhibit amyloid protein aggregation. The inhibitory activity of the invention compounds has been determined in several biological assays routinely utilized by those skilled in the art to measure such amyloid inhibition. Representative invention compounds have been evaluated in the following amyloid assays.

1. BASSR (Beta-Amyloid Self-Seeding Radioassay)

An assay for inhibitors of self-seeded amyloid fibril growth

Materials:

Stock Solutions:

Assay Buffer—50 mM sodium phosphate, pH 7.5, 100 mM NaCl, 0.02% NaN₃, 1 M urea (filter and store at 4° C.)

Soluble Aβ(1-40) peptide (Bachem, Torrance, Calif.)—2.2 mg/mL in deionized H₂O (is stored in aliquots at −20° C.; is kept on ice when thawed) will self-seed after 1 week storage. Typically, the solution is stored until no lag phase is seen in the assay.

¹²⁵I-labeled Aβ(1-40)—150K to 350K cpm/μL in 100% acetonitrile—0.1% trifluoroacetic acid (TFA)—1% β-mercaptoethanol (aliquots stored at −20° C.). ¹²⁵I-labeled Aβ(1-40) is made in accordance with the procedure set forth by LeVine H., III, in Neurobiol. Aging, 1995;16:755, which is hereby incorporated by reference, or this reagent may be purchased from Amersham, Arlington Heights, Ill.

Final assay conditions: 30 μM soluble Aβ(1-40) in deionized water in assay buffer+20 K to 50 K cpm ¹²⁵I-labeled Aβ(1-40) per assay. Compound to be tested is dissolved in dimethylsulfoxide (DMSO), typically 5 to 50 mM stock, such that the final concentration of DMSO is <1% v/v in the assay.

Assay: Reaction mixture for 50 assays (on ice) is comprised of 0.1 to 0.2 μL of ¹²⁵I-labeled A¹²⁵I-labeled Aβ(1-40)+1 μL of soluble Aβ(1-40)+13.5 μL assay buffer per assay. The following are the amounts of the components of the reaction mixture sufficient for 50 assay wells.

5-10 μL ¹²⁵I-labeled Aβ(1-40) dried down

675 μL assay buffer

50 μL soluble Aβ(1-40)

Assay Method:

1) The reaction mixture of above is prepared by mixing components and storing on ice.

2) 14.5 μL of the reaction mixture is pipetted into each of 50 wells on a polypropylene U-bottom 96-well microtiter plate on ice (Costar 3794).

3) 1.7 μL of diluted compound to be tested is added to each well in a column of eight, including solvent control. Serial 3-fold dilutions from 1 mM (100 μM final) in assay buffer−urea=7 dilutions+zero. Each 96-well plate can therefore accommodate 11 samples+1 Congo Red control (0.039-5 μM final in 2-fold steps).

4) The plate with aluminum film (Beckman 538619) is sealed and incubated for 10 minutes on ice.

5) The temperature is raised to 37° C. and incubated for 3 to 5 hours (depending on the lot of the peptide).

6) The aluminum film is removed and 200 μL/well of ice cold assay buffer with urea is added. The radiolabeled fibrils are collected by vacuum filtration through 0.2 μm pore size GVWP filters in 96-well plates (Millipore MAGV N22, Bedford, Mass.). The radioactivity of the filters is determined by using standard methods well-known to those skilled in the art.

2. BASST (Beta-Amyloid Self-Seeding, ThioflavinT)

An assay for inhibitors of self-seeded arnyloid fibril growth

Materials:

Stock Solutions:

Assay Buffer—50 mM sodium phosphate, pH 7.5, 100 mM NaCl, 0.02% NaN₃, 1 M urea (filter and store at 4° C.)

Soluble Aβ (1-40)—2.2 mg/mL in deionized H₂O (is stored in aliquots at −20° C.; is kept on ice when thawed) will self-seed after 1 week storage. Typically, the solution is stored until no lag phase is seen in the assay.

Final assay conditions: 30 μM soluble Aβ(1-40) in deionized water in assay buffer. Compound to be tested is dissolved in DMSO, typically 5 to 50 mM stock, such that the final concentration of DMSO is <1% v/v in the assay.

Assay: Reaction mixture for 50 assays (on ice) is comprised of 1 μL of soluble Aβ(1-40)+13.5 μL assay buffer per assay. The following are the amounts of the components of the reaction mixture that result in each of the 50 assay wells.

50 μL soluble Aβ(1-40)

675 μL assay buffer

Assay Method:

1) The reaction mix above is prepared by mixing the components and storing on ice.

2) 14.5 μL of reaction mixture is pipetted into each of 50 wells of a polystyrene U-bottom, 96-well microtiter plate (Corning 25881-96) on ice.

3) 1.7 μL of diluted compound to be tested is added to each well in a column of eight, including solvent control. Serial 3-fold dilutions from 1 mM (100 μM final) in assay buffer−urea=7 dilutions+zero. Each 96-well plate can therefore accommodate 11 samples+1 Congo Red control (0.039-5 μM final in 2-fold steps).

4) The plate with aluminum film is sealed and incubated for 10 minutes on ice.

5) The temperature is raised to 37° C. and incubated for 3 to 5 hours (depending on the lot of the peptide).

6) The aluminum film is removed and 250 μL/well of 5 μM thioflavin T (ThT) [T-3516, Sigma-Aldrich] is added in 50 mM glycine-NaOH, pH 8.5. Fluorescence is read on a plate reader (ex=440 nm/20 nm; em=485 nm/20 nm) within 5 minutes.

3. BAPA (Beta-Amyloid Peptide Aggregation)

This assay is used to provide a measure of inhibition by a compound against the aggregation behavior of the beta amyloid peptide.

The purpose of this assay is to provide a higher volume method of assaying the amount of beta amyloid aggregation using an endpoint assay based on filtration. In this assay, hexafluoroisopropanol (HFIP) is used to break down the initial amyloid peptide to a monomer state and a concentration of 33 μM is used, a concentration which is high enough so that aggregation will occur at pH 6.0 in several hours.

Method:

β-Amyloid Peptide Aggregation, pH 6.0 (BAPA)

To a 96-well plate (Costar 3794) is added 25 μL 50 mM Phosphate Buffer (pH 6.0), 10 μL 0.5 mg/mL Aβ (1-40) peptide in 20% HFIP+0.1 μL/assay radioiodinated ¹²⁵I Aβ (1-40) [¹²⁵I Aβ(1-40)], and 1 μL of the compound to be tested, starting at 50 mM with a concentration of DMSO <1%. The reaction is incubated for 2 to 4 hours at room temperature. The reaction is stopped with 200 μL of 50 mM phosphate buffer, pH 6.0, and filtered through a 0.2 μm 96-well filter plate (Millipore MAGU N22). The filter plate is washed with 100 μL of the same phosphate buffer. Aggregation is detected on a Microbeta counter after impregnating the filters with Meltilex (1450-441) and is corrected for background.

4. Batym Assay

Methods:

Required Aβ (1-42) (California Peptide) is dried from its hexafluoroisopropanol (HFIP) stock solution. The Aβ (1-42) is dissolved in DMSO and then mixed with phosphate buffered saline (PBS) (pH 7.4). The mixed Aβ (1-42) solution is filtered with a GVWP 0.22 μm syringe filter (Millipore, Bedford, Mass.). The compound to be tested in DMSO (50 times concentrate) is put into each well (0.5 μL/well) of a 96-well plate. The Aβ(1-42) solution is added into each well (25 μL/well). The plate is centrifuged at 1000 g for 5 minutes and incubated at 37° C. for 1 day (Aβ 1-42; final concentration 100 μM).

After incubation Thioflavin T (ThT) (30 μM) solution in glycine-NaOH buffer (pH 8.5, 50 mM) is added into each well (250 μL/well), fluorescence is measured (ex=440/20 nm, em=485/20 nm) using a fluorescence plate reader. The inhibitory activity is calculated as the reduction of fluorescence with the following formula:

Inhibition(%)={(F(Aβ)−F(Aβ+compound)}/{F(Aβ)−F(solvent−compound)}×100.

The IC₅₀s are calculated by a curve-fitting program using the equation given below. The data is obtained from two different experiments in triplicate.

F(x)=100−100/{1+(IC₅₀/10^{x})ₙ};

x=concentration of tested compound (M),

IC₅₀=(M),

n=Hill coefficient.

The results of these assays for compounds of the present invention are shown in Table 2.

4-[4-(3,4-Dichloro-benzylidene)-5-oxo-4,5-dihydro-oxazol-2-yl]-benzoic acid;

3-[2-(3,4-Dichloro-phenyl)-5-oxo-oxazol-4-yildenemethyl]-benzoic acid;

(4-Carboxy-phenyl)-{4-[4-(3,4-dichloro-benzylidene)-5-oxo-4,5-dihydro-oxazol-2-yl]-phenyl}-ammonium;

2-[4-(3,4-Dichloro-benzylidene-5-oxo-4,5-dihydro-oxazol-2-yl]-benzoic acid;

2-[2-(3,4-Dichloro-phenyl)-5-oxo-oxazol-4-yildenemethyl]-benzoic acid;

(2-Carboxy-phenyl)-{4-[4-(3,4-dichloro-benzylidene)-5-oxo-4,5-dihydro-oxal-2-yl]-phenyl}-ammonium;

3-[4-(3,4-Dichloro-benzylidene)-5-oxo-4,5-dihydro-oxazol-2-yl]-benzoic acid;

(3-Carboxy-phenyl)-{4-[4-(3,4-dichloro-benzylidene)-5-oxo-4,5-dihydro-oxazol-2-yl]-phenyl}-ammonium;

4-[2-(3,4-Dichloro-phenyl)-5-oxo-oxazol-4-ylidenemethyl]-benzoic acid;

and

4-(4-Chloro-3-trifluoromethyl-benzylidene)-2-phenyl-4H-oxazol-5-one.

The data in Table 2 establishes the representative invention compounds are active in standard assays used to measure inhibition of protein aggregation. The compounds are thus useful to clinically inhibit amyloid protein aggregation and to image amyloid deposits for diagnostic use. The compounds will be used in the form of pharmaceutical formulations, and the following examples illustrate typical compositions.

TABLE 2

| Example | | | | BASSR | BASST | BATYM |
|---|---|---|---|---|---|---|
| No. | Compound | R₁ | R₂ | (IC₅₀ = μM) | (IC₅₀ = μM) | (IC₅₀ = μM) |
| 1 | 4-Benzylidene-2-phenyl-4H-oxazol-5-one | H | H | >100 | >100 | — |
| 2 | 4-1,3-Benzodioxol-5-ylmethylene-2-phenyl- 4H-oxazol-5-one | 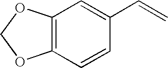 | H | 21 | | 100, >100 |
| 3 | 4-(2-Methoxy-3,5-dinitro-benzylidene)-2- | 2-OMe | H | >100 | 7, 7 | |
| | phenyl-4H-oxazol-5-one | 3,5-(NO₂)₂ | | | | |
| 4 | 5(4H)-Oxazolone,4-[(3,4- | 3,4-(OMe)₂ | H | 7, 8 | >100 | 100, >100 |
| | dimethoxyphenyl)methylene]-2-phenyl- | | | | | |
| 5 | 4-Benzylidene-2-(4-methoxy-phenyl)-4H- | H | 4-OMe | 16 | >100, 0.7 | — |
| | oxazol-5-one | | | | | |
| 6 | 4-(4-Dimethylamino-naphthalen-1- ylmethylene)-2-phenyl-4H-oxazol-5-one | 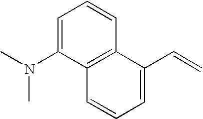 | >100 | 2.6 | 14.5 | |
| 7 | 4-(4-Dipropylamino-benzylidene)-2-phenyl-4H- | 4-dipropylamino | H | >100 | 8 | 2.42, 2.36 |
| | oxazol-5-one | | | | | |
| 8 | 4-(3-Methoxy-benzylidene)-2-phenyl-4H-oxazol- | 3-OMe | H | >100 | 7.0 | — |
| | 5-one | | | | | |
| 9 | 4-(2,3-Dimethoxy-benzylidene)-2-(4-nitro- | 2,3-(OMe)₂ | 4-NO₂ | >100 | 12 | — |
| | phenyl)-4H-oxazol-5-one | | | | | |
| 10 | 4-(3,4-Dimethoxy-benzylidene)-2-(2-iodo-phenyl)- | 3,4-(OMe)₂ | 2-I | >100 | 15 | |
| | 4H-oxazol-5-one | | | | | |
| 11 | 4-(3,4-Dichloro-benzylidene)-2-phenyl-4H-oxazol- | 3,4-Cl₂ | H | 21 | 30 | — |
| | 5-one | | | | | |
| 12 | 4-(3,4-Dimethyl-benzylidene)-2-phenyl-4H- | 3,4-Me2 | H | >100 | | |
| | oxazol-5-one | | | | | |
| 13 | 4-(3,4-Dichloro-benzylidene)-2-(4-nitro-phenyl)- | 3,4-Cl₂ | 4-NO₂ | 70 | 90 | |
| | 4H-oxazol-5-one | | | | | |
| 14 | 4-(4-Fluoro-3-trifluoromethyl-benzylidene)-2- | 3-CF_{3, 4-F} | H | >100 | >100 | |
| | phenyl-4H-oxazol-5-one | | | | | |

### EXAMPLE 1

Preparation of 4-(4-Nitro-benzylidene)-2-phenyl-4H-oxazol-5-one

A mixture of 4-nitrobenzaldehyde (10.0 g, 0.066 mol), hippuric acid (13.04 g, 0.072 mol), sodium acetate (5.52 g, 0.067 mol), and acetic anhydride (18.68 mL) was heated to reflux for 3 hours (110° C.). After cooling to room temperature for 72 hours, the precipitate was triturated with ethanol and washed with water, and then dried under vacuum to give the product as a brown solid, 7.85 g (0.0266 mol, 40%); mp 226-228° C.

Analysis of C₁₆H₁₀N₂O₄.0.4 mol H₂O: C, 63.74; H, 3.61; N, 9.28. Found: C, 63.86; H, 3.56; N, 9.56.

### EXAMPLE 2

Preparation of 4-(3,4-Dimethoxy-benzylidene)-2-phenyl-4H-oxazol-5-one

A mixture of 3,4-dimethylbenzaldehyde (1.66 g, 0.10 mol), hippuric acid (1.97 g, 0.11 mol), sodium acetate (0.84 g, 0.102 mol), and acetic anhydride (2.83 mL) was heated to reflux for 3 hours (110° C.). After cooling to room temperature, the precipitate was triturated with ethanol and washed with water, and then dried under vacuum to give the product as a yellow solid, 1.12 g (0.003 mol, 36%); mp 146-148° C.

Analysis of C₁₈H₁₅NO₄.0.25 mol H₂O: C, 68.89; H, 4.98; N, 4.46. Found: C, 68.88; H, 4.85; N, 4.38.

### EXAMPLE 3

Preparation of 4-(4-Dimethylamino-naphthalen-1-ylmethylene)-2-phenyl-4H-oxazol-5-one

A mixture of 4-dimethylamino-1-napthaldehyde (1.04 g, 0.005 mol), hippuric acid (1.03 g, 0.0057 mol), sodium acetate (0.44 g, 0.0053 mol), and acetic anhydride (1.47 mL) was heated to reflux for 4 hours (110° C.). After cooling to room temperature, the precipitate was triturated with ethanol, washed with water, and then dried under vacuum. The crude solid was purified via MPLC (10% EtOAc/hexanes) to give the product as a red/brown solid, 100 mg (0.178 mol, 15.9%); mp 103-106° C.

Analysis of C₂₂H₁₈N₂O₂.0.5 mol ethyl acetate: Calcd: C, 74.59; H, 5.74; N, 7.25. Found: C, 73.66; H, 5.83; N, 7.33.

### EXAMPLE 4

Preparation of 4-(3,5-Dichloro-2-hydroxy-benzylidene)-2-phenyl-4H-oxazol-5-one

A mixture of 3,5-dichlorosalicylaldehyde (5.0 g, 0.026 mol), hippuric acid (5.16 g, 0.029 mol), sodium acetate (2.19 g, 0.0267 mol) and acetic anhydride (1.47 mL) was heated to reflux for 4 hours (110° C.). After cooling to room temperature, the precipitate was triturated with ethanol, washed with water, and then dried under vacuum. The crude solid was purified via MPLC (10% EtOAc/hexanes) to give the product as a red/brown solid, 100 mg (0.178 mol, 15.9%); mp 210-212° C.

Analysis of C₁₆H₉NO₃Cl₂.0.14 mol H₂O.0.44 mol ethyl acetate: Calcd: C, 56.82; H, 3.44; N, 3.73. Found: C, 56.75; H, 3.52; N, 3.51.

### EXAMPLE 5

Preparation of 4-(2,3-Dihydroxy-benzylidene)-2-phenyl-4H-oxazol-5-one

A mixture of 2,3-dihydroxybenzaldehyde (5.0 g, 0.036 mol), hippuric acid (7.13 g, 0.040 mol), sodium acetate (3.03 g, 0.037 mol), and acetic anhydride (10.25 mL) was heated to reflux for 24 hours (110° C.). After cooling to room temperature, the solution was concentrated, and then diluted with ethyl acetate and washed with water. The organic layer was dried with Mg₂SO₄, concentrated, and dried under vacuum. The dark brown solid was purified via MPLC (30% EtOAc/hexanes) to give the product as a yellow/brown solid, 0.74 mg (0.003 mol, 7.3%); mp 210-212° C.

Analysis of C₁₆H₁₁NO₄.0.04 mol H₂O.0.25 mol ethyl acetate: Calcd: C, 67.16; H, 4.34; N, 4.61. Found: C, 67.17; H, 4.23; N, 4.33.

### EXAMPLE 6

Preparation of 4-(4-Phenoxy-benzylidene)-2-phenyl-4H-oxazol-5-one

A mixture of 4-phenoxybenzaldehdye (2.06 g, 0.010 mol), hippuric acid (2.06 g, 0.014 mol), sodium acetate (0.88 g, 0.011 mol), and acetic anhydride (2.94 mL) was heated to reflux for 4 hours (110° C.) during which an additional 0.50 g of hippuric acid was added. After cooling to room temperature, the precipitate was triturated with ethanol, washed with water, and then dried under vacuum to give the product as a yellow solid, 1.55 g (0.005 mol, 43.6%); mp 156-158° C.

Analysis of C₂₂H₁₅NO₃: Calcd: C, 77.41; H, 4.43; N, 4.10. Found: C, 77.02; H, 4.39; N, 4.03.

### EXAMPLE 7

Preparation of 4-(4-Dimethylamino-benzylidene)-2-phenyl-4H-oxazol-5-one

A mixture of 4-dimethylaminobenzaldehyde (5.40 g, 0.036 mol), hippuric acid (7.13 g, 0.039 mol), sodium acetate (3.03 g, 0.036 mol) and acetic anhydride (10.25 mL) was heated to reflux for 20 hours (110° C.). After cooling to room temperature, a precipitate formed which was dissolved in ethyl acetate and water and then filtered. The solid was collected and dried under vacuum to give a pink/red solid. This solid was stirred in ether, filtered and dried under vacuum overnight to give 2.06 g of red crystals (0.007 mol, 21.29%); mp 216-217° C.

Analysis of C₁₈H₁₆N₂O₂: Calcd: C, 73.96; H, 5.52; N, 9.58; Found: C, 73.62; H, 5.46; N, 9.55

### EXAMPLE 8

Preparation of 4-Naphthalen-1-ylmethylene-2-phenyl-4H-oxazol-5-one

A mixture of 1-napthaldehyde (1.63 g, 0.010 mol), hippuric acid (2.06 g, 0.011 mol), sodium acetate (0.88 g, 0.011 mol), and acetic anhydride (2.94 mL) was heated to reflux for 0.5 hour (110° C.). After cooling to room temperature, the precipitate was triturated with ethanol, washed with water, and then dried under vacuum. The product was a yellow-orange solid, 879 mg (0.003 mol, 22.19%); mp 167-168° C.

Analysis of C₂₀H₁₃N₁O₂.0.03 mol H₂O: Calcd: C, 80.11; H, 4.39; N, 4.67. Found: C, 80.09; H, 4.07; N, 4.68.

### EXAMPLE 9

Preparation of 4-Naphthalen-2-ylmethylene-2-phenyl-4H-oxazol-5-one

A mixture of 2-napthaldehyde (0.81 g, 0.005 mol), hippuric acid (1.03 g, 0.006 mol), sodium acetate (0.44 g, 0.005 mol), and acetic anhydride (1.5 mL) was heated to reflux for 0.5 hour (110° C.). After cooling to room temperature, the precipitate was triturated with ethanol, washed with water, and then dried under vacuum. The product was a yellow solid, 929 mg (0.003 mol, 59.9%); mp 147-148° C.

Analysis of C₂₀H₁₃N₁O₂.0.06 mol H₂O: Calcd: C, 79.96; H, 4.40; N, 4.66. Found: C, 79.94; H, 4.13; N, 4.67

### EXAMPLE 10

Preparation of 4-(2,3-Dimethoxy-benzylidene)-2-(2-iodo-phenyl)-4H-oxazol-5-one

A mixture of 2,3-dimethoxybenzaldehyde (1.66 g, 0.010 mol), 2-iodohippuric acid (3.36 g, 0.011 mol), sodium acetate (0.84 g, 0.010 mol), and acetic anhydride (2.83 mL) was heated to reflux for 50 minutes (110° C.). After cooling to room temperature, the precipitate was triturated with ethyl acetate and methanol, filtered, and then dried under vacuum. The product was a yellow solid, 915 mg (0.002 mol, 21%); mp 164-166° C.

Analysis of C₁₈H₁₄₁N₁04-0.06 mol H₂O: Calcd: C, 49.55; H, 3.26; N, 3.21. Found: C, 49.56; H, 3.13; N, 3.08

### EXAMPLE 11

Preparation of 4-(2-Methoxy-benzylidene)-2-phenyl-4H-oxazol-5-one

A mixture of 2-methoxybenzaldehyde (1.36 g, 0.010 mol), hippuric acid (1.97 g, 0.011 mol), sodium acetate (0.84 g, 0.010 mol) and acetic anhydride (2.83 mL) was heated to reflux for 0.5 hour (110° C.). After cooling to room temperature, the precipitate was triturated with ethanol, washed with water, and then dried under vacuum. The product was a yellow solid, 1.39 g (0.005 mol, 50%); mp 159-161° C.

Analysis of C₁₇H₁₃N₁O₃: Calcd: C, 73.11; H, 4.69; N, 5.01. Found: C, 71.74; H, 4.65; N, 4.91.

### EXAMPLE 12

Preparation of 4-(3-Methoxy-benzylidene)-2-phenyl-4H-oxazol-5-one

A mixture of 3-methoxybenzaldehyde (1.2 mL, 0.010 mol), hippuric acid (1.97 g, 0.011 mol), sodium acetate (0.84 g, 0.010 mol), and acetic anhydride (2.83 mL) was heated to reflux for 0.5 hour (110° C.). After cooling to room temperature, the precipitate was triturated with ethanol, washed with water, and then dried under vacuum. The product was a yellow solid, 0.931 g (0.003 mol, 33.4%); mp 100-102° C.

Analysis of C₁₇H₁₃N₁O₃: Calcd: C, 73.11; H, 4.69; N, 5.01. Found: C, 72.71; H, 4.68; N, 4.78.

### EXAMPLE 13

Preparation of 4-(4-Methoxy-benzylidene)-2-phenyl-4H-oxazol-5-one

A mixture of 4-methoxybenzaldehyde (1.2 mL, 0.010 mol), hippuric acid (1.97 g, 0.011 mol), sodium acetate (0.84 g, 0.010 mol), and acetic anhydride (2.83 mL) was heated to reflux for 0.75 hour (110° C.). After cooling to room temperature, the precipitate was triturated with ethanol, with water, and then dried under vacuum. The product was a yellow solid, 1.29 g (0.005 mol, 46.1%); mp 157-158° C.

Analysis of C₁₇H₁₃N₁O₃: Calcd: C, 73.11; H, 4.69; N, 5.01. Found: C, 72.80; H, 4.59; N, 4.90.

### EXAMPLE 14

Preparation of (2,3-Dimethoxy-benzylidene)-2-phenyl-4H-oxazol-5-one

A mixture of 2,3-dimethoxybenzaldehyde (1.66 g, 0.010 mol), hippuric acid (1.97 g, 0.011 mol), sodium acetate (0.84 g, 0.010 mol), and acetic anhydride (2.83 mL) was heated to reflux for 0.5 hour (110° C.). After cooling to room temperature, the precipitate was triturated with ethanol, washed with water, and then dried under vacuum. The product was a yellow solid, 1.35 g (0.004 mol, 43.7%); mp 165-167° C.

Analysis of C₁₈H₁₅N₁O₄.0.24 mol H₂O: Calcd: C, 68.93; H, 4.97; N, 4.47. Found: C, 69.89; H, 4.89; N, 4.53.

### EXAMPLE 15

Preparation of 4-(3,4-Dimethoxy-benzylidene)-2-(2-hydroxy-phenyl)-4H-oxazol-5-one

A mixture of 3,4-dimethoxybenzaldehyde (1.66 g, 0.010 mol), o-hydroxyhippuric acid (2.15 g, 0.011 mol), sodium acetate (0.84 g, 0.010 mol), and acetic anhydride (2.83 mL) was heated to reflux for 1 hour (110° C.). After cooling to room temperature, the precipitate was triturated with ethanol, washed with water, and then dried under vacuum. The product was an orange-yellow solid, 1.18 g (0.004 mol, 36%); mp 209-210° C.

Analysis of C₁₈H₁₅N₁O₅0.05 mol H₂O: Calcd: C, 66.27; H, 4.67; N, 4.29. Found: C, 66.26; H, 4.64; N, 4.30.

### EXAMPLE 16

Preparation of 4-(2,3-Dimethoxy-benzylidene)-2-(4-nitro-phenyl)-4H-oxazol-5-one

A mixture of 2,3-dimethoxybenzaldehyde (7.64 g, 0.046 mol), p-nitrohippuric acid (11.34 g, 0.051 mol), sodium acetate (3.85 g, 0.047 mol), and acetic anhydride (13.0 mL) was heated to reflux for 6.5 hours (110° C.). After cooling to room temperature, the precipitate was washed with ethyl acetate, and then dried under vacuum. The product was an orange-yellow solid, 7.79 g (0.022 mol, 47.8%); mp 203-208° C.

Analysis of C₁₈H₁₄N₂O₆: Calcd: C, 61.02; H, 3.98; N, 7.91. Found: C, 56.72; H, 3.85; N, 7.90.

### EXAMPLE 17

Preparation of 4-(3,4-Dimethoxy-benzylidene)-2-(2-iodo-phenyl)-4H-oxazol-5-one

A mixture of 3,4-dimethoxybenzaldehyde (0.55 g, 0.003 mol), o-iodohippuric acid (1.12 g, 0.004 mol), sodium acetate (0.28 g, 0.003 mol), and acetic anhydride (0.94 mL) was heated to reflux for 1 hour (110° C.). After cooling to room temperature, the precipitate was triturated with ethyl acetate, washed with water, and then dried under vacuum. The product was an orange solid, 0.396 g (0.009 mol, 27%); mp 200-202° C.

Analysis of C₁₈H₁₄N₁O₄I: Calcd: C, 49.68; H, 3.24; N, 3.22. Found: C, 49.68; H, 3.20; N, 3.18.

### EXAMPLE 18

Preparation of 4-(3,4-Dimethoxy-benzylidene)-2-(4-nitro-phenyl)-4H-oxazol-5-one

A mixture of 3,4-dimethoxybenzaldehyde (8.3 1 g, 0.05 mol), p-nitrohippuric acid (12.33 g, 0.055 mol), sodium acetate (4.18 g, 0.051 mol), and acetic anhydride (14.15 mL) was heated to reflux for 0.5 hour (110° C.). After cooling to room temperature, the precipitate was triturated with ethyl acetate, filtered, and then dried under vacuum. A sample was triturated with 50:50 acetone/ethyl acetate, filtered, and dried to a red-orange solid, 0.023 g (0.0006 mol); mp 265-267° C.

Analysis of C₁₈H₁₄N₂O₆: Calcd: C, 61.02; H, 3.98; N, 7.91. Found: C, 56.01; H, 3.72; N, 8.06.

### EXAMPLE 19

Preparation of 4-(2-Hydroxy-naphthalen-1-ylmethylene)-2-phenyl-4H-oxazol-5-one

A mixture of 2-hydroxy-1-napthaldehyde (1.0 g, 0.006 mol), hippuric acid (1.14 g, 0.006 mol), sodium acetate (0.49 g, 0.006 mol), and acetic anhydride (1.64 mL) was heated to reflux for 15 minutes when an additional 1 mL of acetic anhydride was added and heated for an additional 0.5 hour (110° C.). After cooling to room temperature, the precipitate was triturated with ethanol and then dried under vacuum to yield a white solid, 834 mg (0.003 mol, 45.5%); mp 228-229° C.

Analysis of C₂₀H₁₃N₁O₃: Calcd: C, 76.18; H, 4.16; N, 4.44. Found: C, 75.20; H, 3.76; N, 4.39.

### EXAMPLE 20

Preparation of 4-(4-Nitro-naphthalen-1-ylmethylene)-2-phenyl-4H-oxazol-5-one

Step A: Preparation of Tris (benzotriazol-1-yl)methane

A mixture of benzotriazole (60.0 g, 0.504 mol), chloroform (50.5 mL, 0.970 mol), tetrabutylammonium bromide (1.61 g, 5.00 mol), and 40% NaOH in water (50 mL) were refluxed under N₂ for 72 hours. A solid formed and the solution was cooled to room temperature and filtered through celite. The solid that was formed in the reaction mixture was extracted with chloroform, concentrated, and dried in vacuo to yield a light-brown solid. This solid was triturated with 150 mL of methanol, filtered, and dried under vacuum to give a light-brown solid, 24.5 g (0.067 mol, 46.3%; mp 194-195° C.).

Analysis of C₁₉H₁₃N₉: Calcd: C, 62.12; H, 3.57; N, 34.31. Found: C, 61.46; H, 3.40; N, 33.76.

Step B: Preparation of para-[Bis(benzotriazol-1-yl)methyl]nitronapthalene

Under nitrogen conditions, 1-nitronapthalene (11.31 g, 0.065 mol), tris(benzotriazol-1-yl)methane (24.00 g, 0.065 mol), and potassium hydroxide (22.94 g, 0.408 mol) in dimethylsulfoxide (327 mL) were stirred overnight. The solution was then poured into 588 mL of 1N HCl. The solution was stirred and a tan precipitate formed. The solution was extracted with chloroform, and the combined organics were concentrated to approximately 250 mL. This solution was then washed with water and dried with Mg₂SO₄, filtered, and concentrated to a dark sticky solid. Purification via MPLC (40% ethyl acetate/hexanes) yielded 12.88 g (0.031 mol, 46.8%) of product; mp 212-213° C.

Analysis of C₂₃H₁₅N₇O₂: Calcd: C, 65.55; H, 3.59; N, 23.27. Found: C, 64.41; H, 3.43; N, 22.7.

Step C: Preparation of 4-Nitro-naphthalene-1-carbaldehyde

A mixture of para-[Bis(benzotriazol-1-yl)methyl]nitronapthalene (3.37 g, 0.008 mol), zinc bromide (3.60 g, 0.016 mol), and dioxane (100 mL) were refluxed at approximately 120° C. for 30 minutes. Concentrated HCl (7 mL) was then added and the mixture was refluxed for an additional 4 hours. The mixture was then cooled to room temperature, and the solvent was concentrated in vacuo. The solution was taken up in 200 mL of water and extracted with dichloromethane (3×100 mL). The organic layers were washed with 5% NaOH (2×60 mL), dried over Mg₂SO₄, filtered, and concentrated to a red oil that crystallized with cooling. This solid was dried for 24 hours. Purification via MPLC (40% ethyl acetate/hexanes and then 25% ethyl acetate/hexanes) yielded an orange solid, 0.79 g (0.004 mol, 49%); mp 96-99° C.

Analysis of C₁₁H₇N₁O₃: Calcd: C, 65.67; H, 3.51; N, 6.96. Found: C, 65.44; H, 3.60; N, 6.89.

Step D: Preparation of 4-(4-Nitro-naphthalen-1-ylmethylene)-2-phenyl-4H-oxazol-5-one

A mixture of 4-nitro-naphthalene-1-carbaldehyde (0.72 g, 0.004 mot), hippuric acid (0.71 g, 0.004 mol), sodium acetate (0.30 g, 0.004 mol), and acetic anhydride (1.02 mL) was heated to reflux. An additional amount of acetic anhydride (1.5 mL) was then added and heated to 110% for an additional 0.75 hour. After cooling to room temperature, the precipitate was triturated with ethanol, washed with water, and dried under vacuum to give a brown solid. This crude brown solid was dissolved in hot ethyl acetate, filtered hot, and recrystallized to yield a yellow-orange solid, 440 mg (0.001 mol, 35.5%); mp not available.

Analysis of C₂₀H₁₂N₂O₄: Calcd: C, 69.77; H, 3.51; N, 8.14. Found: C, 69.73; H, 3.49; N, 8.13.

### EXAMPLE 21

Preparation of 2-(4-Amino-phenyl)-4-(3,4-dimethoxy-benzylidene)-4H-oxazol-5-one

4-(3,4-Dimethoxy-benzylidene)-2-(4-nitro-phenyl)-4H-oxazol-5-one (1.0 g, 0.003 mol) was dissolved in trifluoroacetic acid (20 mL), and iron powder (reduced) (1.83 g, 0.032 mol) was added. The reaction mixture was stirred under nitrogen for 4 hours, and the iron was then removed. The solvent was concentrated and the residue was then taken up in ethyl acetate (150 mL), washed with saturated sodium bicarbonate solution (2×50 mL), dried with sodium sulfate, filtered, concentrated in vacuo, and dried under vacuum to give the product as a red-orange solid, 0.43 g (0.001 mol, 47%); mp 196-200° C.

Analysis of C₁₈H₁₆N₂O₄.0.9 mol H₂O.0.2 mol ethyl acetate: Calcd: C, 63.04; H, 5.46; N, 7.82. Found: C, 63.26; H, 4.80; N, 7.48.

### EXAMPLE 22

Preparation of 2-Phenyl-4-(2,3,4-trihydroxy-benzylidene)-4H-oxazol-5-one

A mixture of 2,3,4-trihydroxybenzaldeheyde (5.0 g, 0.032 mol), hippuric acid (6.39 g, 0.0356 mol), sodium acetate (2.68 g, 0.032 mol) and acetic anhydride (9.05 mL) was heated to reflux for 8 hours (110° C.). After cooling to room temperature, the precipitate was triturated with ether, filtered, and dried under vacuum, 0.71 g (0.0024 mol, 7.46%); mp 153-156° C.

Analysis of C₁₆H₁₁N₁O₅.4.5 mol H₂O: C, 50.79; H, 5.33; N, 3.70. Found: C, 50.82; H, 4.43; N, 4.83.

### EXAMPLE 23

Preparation of 4-(4-Dipentylamino-benzylidene)-2-phenyl-4H-oxazol-5-one

Step A: Preparation of 4-Dipentylamino-benzaldehyde

4-Fluorobenzaldhyde (4.9 g, 0.040 mol), dipentylamine (12.58 g, 0.080 mol), and potassium carbonate (6.62 g, 0.048 mol) were heated to 90° C. in Dimethylamine (10 mL) for 3 days with vigorous stirring. The reaction mix was cooled to room temperature, diluted with water, and extracted with ether. The combined organics were dried with Mg₂SO₄, filtered, and concentrated. The resultant was purified via MPLC (5% ethyl acetate/hexanes) to yield a yellow oil, (1.65 g) (24% yield).

Step D: Preparation of 4-(4-Dipentylamino-benzylidene)-2-phenyl-4H-oxazol-5-one

A mixture of 4-Dipentylamino-benzaldehyde (1.0 g, 0.005 mol), hippuric acid (0.978 g, 0.006 mol), sodium acetate (0.415 g, 0.005 mol), and acetic anhydride (1.40 mL) was heated to reflux for 3 hours (110° C.). After cooling to room temperature for 2 days, the mixture was extracted with water and ethyl acetate. The organic layers were combined, dried with sodium sulfate, filtered, and concentrated. The crude oil was purified via MPLC (5% ethyl acetate/hexanes) to give the product as a red oil, 0.350 g (0.8650 mmol, 20.58%).

Analysis of C₂₆H₃₂N₂O₂.0.25 mol H₂O: C, 76.33; H, 8.00; N, 6.85. Found: C, 76.32; H, 8.00; N, 6.23.

### EXAMPLE 24

Preparation of 4-(3,4-Dichloro-benzylidene)-2-phenyl-4H-oxazol-5-one

A mixture of 3,4-dichlorobenzaldeheyde (5.0 g, 0.028 mol), hippuric acid (5.63 g, 0.031 mol), sodium acetate (2.39 g, 0.029 mol), and acetic anhydride (8.07 mL) was heated to reflux for 3 hours (110° C.) with an additional 8 mL of acetic anhydride added. After cooling to room temperature, the precipitate was triturated with ethanol, washed with water, and the resultant solid was dried under vacuum for 24 hours. Purification via MPLC (2×50:50 etyhl acetate/hexanes) followed by HPLC (25% to 100% acetonitrile with 3.0% α-propanol and water with 3.0%, N-propanol) and column purification of a 300 mg quantity gave 80 mg of yellow solid; mp 157, 167, 170° C.

Analysis of C₁₆H₉Cl₂N₁O₂.0.06 mol H₂O: C, 60.20; H, 2.88; N, 4.39. Found: C, 59.82; H, 2.94; N, 4.16.

### EXAMPLE 25

Preparation of 4-(3,4-Dimethyl-benzylidene)-2-phenyl-4H-oxazol-5-one

A mixture of 3,4-dimethylbenzaldeheyde (5.0 g, 0.037 mol), hippuric acid (7.35 g, 0.041 mol), sodium acetate (2.39 g, 0.029 mol), and acetic anhydride (10.55 mL) was heated to reflux for 3 hours (110° C.). After cooling to room temperature, the precipitate was triturated with ethanol, washed with water, and the resultant solid was dried under vacuum overnight. Purification via MPLC (10% ethyl acetate/hexanes and then 3% ethyl acetate/hexanes) gave a yellow solid 1.3 g (0.005 mol, 12.57%); mp 120-122° C.

Analysis of C₁₈H₁₅N₁O₂.0.1 mol H₂O: C, 77.46; H, 5.49; N, 5.02. Found: C, 77.08; H, 5.66; N, 5.02.

### EXAMPLE 26

Preparation of 4-(4-Fluoro-3-trifluoromethyl-benzylidene)-2-phenyl-4H-oxazol-5-one

A mixture of 4-fluoro-3-trifluormethylbenzaldeheyde (5.0 g, 0.026 mol), hippuric acid (5.13 g, 0.028 mol), sodium acetate (2.18 g, 0.026 mol) and acetic anhydride (7.37 mL) was heated to reflux for 3 hours (110° C.). After cooling to room temperature, the precipitate was triturated with ethanol, washed with water, and the resultant solid was dried under vacuum overnight. The solid was purified via MPLC (0%-10% ethyl acetate/hexanes) to give a yellow solid, 0.105 g (0.0003 mol, 1.2%); mp 192-194° C.

Analysis of C₁₇H₉F₄N₁O₂.0.29 mol H₂O: C, 59.97; H, 2.84; N, 4.11. Found: C, 59.57; H, 2.93; N, 4.00.

### EXAMPLE 27

Preparation of 4-(3,4-Dichloro-benzylidene)-2-(4-nitro-phenyl)-4H-oxazol-5-one

A mixture of 3,4-dichlorobenzaldeheyde (3.0 g, 0.017 mol), hippuric acid (4.23 g, 0.019 mol), sodium acetate (1.43 g, 0.018 mol), and acetic anhydride (4.85 mL) was heated to reflux for 4.5 hours (110° C.). After cooling to room temperature, the precipitate was triturated with ethanol, washed with water, and the resultant solid was dried under vacuum overnight. Purification via MPLC (0%-10% ethyl acetate/hexanes), followed by trituration with ethyl acetate and acetone (50/50) gave a yellow solid, 0.150 g (0.0004 mol., 2.262%); mp 238-242° C.

Analysis of C₁₆H₈Cl₂N₂O₄.0.08 mol H₂O: C, 52.71; H, 2.26; N, 7.68. Found: C, 53.09; H, 2.68; N, 7.12.

### EXAMPLE 28

Tablet Formulation

| Ingredient | Amount |
|---|---|
| Compound of Example 2 | 50 mg |
| Lactose | 80 mg |
| Cornstarch (for mix) | 10 mg |
| Cornstarch (for paste) | 8 mg |
| Magnesium Stearate (1%) | 2 mg |
| | 150 mg |

The compound of Example 1 (Compound of Example 2) with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for prevention of amyloid protein aggregation and treatment of Alzheimer's disease.

### EXAMPLE 29

Parenteral Solution

In a solution of 700 mL of propylene glycol and 200 mL of water for injection is added 20.0 g of Compound of Example 2. The mixture is stirred and the pH is adjusted to 5.5 with hydrochloric acid. The volume is adjusted to 1000 mL with water for injection. The solution is sterilized, filled into 5.0 mL ampoules, each containing 2.0 mL (40 mg of Compound of Example 2), and sealed under nitrogen. The solution is administered by injection to a patient suffering from medullary carcinoma of the thyroid and in need of treatment.

### EXAMPLE 30

Patch Formulation

Ten milligrams of Compound of Example 2 is mixed with 1 mL of propylene glycol and 2 mg of acrylic-based polymer adhesive containing a resinous cross-linking agent. The mixture is applied to an impermeable backing (30 cm²) and applied to the upper back of a patient for sustained release treatment of amyloid polyneuropathy.

The invention and the manner and process of making and using it are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

What is claimed is:

1. A method of treating Alzheimer's disease, the method comprising administering to a patient having Alzheimer's disease a therapeutically effective amount of a compound of Formula I 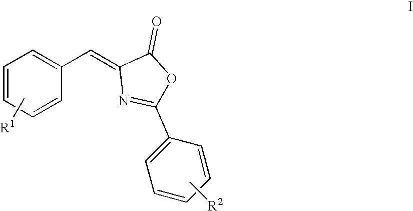
wherein
R¹ is hydrogen, lower alkoxy, hydroxy, aryl, heteroaryl, phenoxy, halogen, hydroxy, cyano, carboxy, alkoxycarbonyl, carbamoyl, sulfamoyl, nitro, trifluoromethyl, amino, mono- or dialkylamino, or lower alkyl or lower alkenyl unsubstituted or substituted with one, two, or three groups independently selected from oxo, halogen, hydroxy, carboxy, carbamoyl, amino, mono- or dialkylamino, or
aryl or heteroaryl optionally substituted independently with up to three groups selected from halogen, lower alkyl, lower alkoxy, hydroxy, carboxy, alkoxycarbonyl, cyano, nitro, trifluoromethyl, amino, mono- or dialkylamino, carbamoyl, carboxyalkyl, alkoxycarbonylalkyl, sulfamoyl, or carbonylamino;
R² is hydrogen, lower alkyl, lower alkoxy, halogen, hydroxy, aryl, heteroaryl, arylalkyl, heteroarylalkyl, arylalkoxy, heteroarylalkoxy, cyano, carboxy, alkoxycarbonyl, carbamoyl, sulfamoyl, nitro, trifluoromethyl, amino, or mono- or dialkylamino.

2. A method according to claim 1, wherein R¹ is hydrogen, halogen, alkoxy, or heteroaryl; and
R² is hydrogen or alkoxy.

3. A method according to claim 1, wherein the compound is selected from:
4-Benzylidene-2-phenyl-4H-oxazol-5-one;
4-(4-Nitro-benzylidene)-2-phenyl-4H-oxazol-5-one;
2-Acetoxy-5-(5-oxo-2-phenyl-oxazol-4-ylidenemethyl)-benzoic acid;
4-1,3-Benzodioxol-5-ylmethylene-2-phenyl-4H-oxazol-5-one;
4-(2-Methoxy-3,5-dinitro-benzylidene)-2-phenyl-4H-oxazol-5-one;
2-Chloro-N,N-dimethyl-5-[1-(5-oxo-2-phenyl-oxazol-4-ylidene)-ethyl]-benzenesulfonamide;
4-Biphenyl-4-ylmethylene-2-phenyl-4H-oxazol-5-one;
4-(3-Ethoxy-benzylidene)-2-phenyl-4H-oxazol-5-one;
4-(3,5-Dibromo-2-hydroxy-benzylidene)-2-phenyl-4H-oxazol-5-one;
4-(4-Ethoxy-benzylidene)-2-phenyl-4H-oxazol-5-one;
4-(2,6-Dichloro-benzylidene)-2-phenyl-4H-oxazol-5-one;
2,2-Dimethyl-5-[4-(5-oxo-2-phenyl-oxazol-4-ylidenemethyl)-phenoxy]-pentanoic acid;
4-(2-Chloro-cyclohex-1-enylmethylene)-2-phenyl-4H-oxazol-5-one;
2-Phenyl-4-pyridin-3-ylmethylene-4H-oxazol-5-one;
4-(4-Hydroxy-benzylidene)-2-phenyl-4H-oxazol-5-one;
4-(3,4-Dimethoxy-benzylidene)-2-phenyl-4H-oxazol-5-one;
4-Benzylidene-2-(4-nitro-phenyl)-4H-oxazol-5-one;
N-[4-(4-Benzylidene-5-oxo-4,5-dihydro-oxazol-2-yl)-phenyl]-acetamide;
4-Benzylidene-2-(4-chloro-phenyl)-4H-oxazol-5-one;
4-Benzylidene-2-p-tolyl-4H-oxazol-5-one;
4-Benzylidene-2-(4-methoxy-phenyl)-4H-oxazol-5-one;
4-Benzo[1,3]dioxol-5-ylmethylene-2-phenyl-4H-oxazol-5-one;
2-(4-Chloro-3-nitro-phenyl)-4-(4-diethylamino-benzylidene)-4H-oxazol-5-one;
4-(4-Dimethylamino-naphthalen-1-ylmethylene)-2-phenyl-4H-oxazol-5-one;
4-(3,5-Dichloro-2-hydroxy-benzylidene)-2-phenyl-4H-oxazol-5-one;
4-(2,3-Dihydroxy-benzylidene)-2-phenyl-4H-oxazol-5-one;
4-(4-Phenoxy-benzylidene)-2-phenyl-4H-oxazol-5-one;
4-(4-Dimethylamino-benzylidene)-2-phenyl-4H-oxazol-5-one;
4-(4-Dimethylamino-benzylidene)-2-phenyl-4H-oxazol-5-one;
4-Naphthalen-1-ylmethylene-2-phenyl-4H-oxazol-5-one;
4-Naphthalen-2-ylmethylene-2-phenyl-4H-oxazol-5-one;
4-(4-Dipropylamino-benzylidene)-2-phenyl-4H-oxazol-5-one;
4-(4-Diethylamino-benzylidene)-2-p-tolyl-4H-oxazol-5-one;
4-(4-Diethylamino-benzylidene)-2-(3,5-dinitro-phenyl)-4H-oxazol-5-one;
4-(2,4-Dichloro-benzylidene)-2-phenyl-4H-oxazol-5-one;
2-(4-Chloro-3-nitro-phenyl)-4-pyridin-4-ylmethylene-4H-oxazol-5-one;
2-(4-Chloro-phenyl)-4-pyridin-2-ylmethylene-4H-oxazol-5-one;
4-(2-Hydroxy-5-nitro-benzylidene)-2-phenyl-4H-oxazol-5-one;
2-(4-Chloro-3-nitro-phenyl)-4-(4-fluoro-benzylidene)-4H-oxazol-5-one;
4-(4-Dimethylamino-benzylidene)-2-(3-nitro-phenyl)-4H-oxazol-5-one;
4-(4-Chloro-benzylidene)-2-(3-nitro-phenyl)-4H-oxazol-5-one;
4-(4-Methoxy-benzylidene)-2-(3-nitro-phenyl)-4H-oxazol-5-one;
4-(4-Diethylamino-benzylidene)-2-(3-nitro-phenyl)-4H-oxazol-5-one;
4-(4-Methyl-benzylidene)-2-(3-nitro-phenyl)-4H-oxazol-5-one;
2-(4-Bromo-phenyl)-4-(4-diethylamino-benzylidene)-4H-oxazol-5-one;
2-(2-Chloro-phenyl)-4-(4-diethylamino-benzylidene)-4H-oxazol-5-one;
2-(3-Nitro-phenyl)-4-pyridin-4-ylmethylene-4H-oxazol-5-one;
4-(2,3-Dimethoxy-benzylidene)-2-(2-iodo-phenyl)-4H-oxazol-5-one;
4-(2-Methoxy-benzylidene)-2-phenyl-4H-oxazol-5-one;
4-(3-Methoxy-benzylidene)-2-phenyl-4H-oxazol-5-one;
4-(4-Methoxy-benzylidene)-2-phenyl-4H-oxazol-5-one;
4-(2,3-Dimethoxy-benzylidene)-2-phenyl-4H-oxazol-5-one;
4-(3,4-Dimethoxy-benzylidene)-2-(2-hydroxy-phenyl)-4H-oxazol-5-one;
4-(2,3-Dimethoxy-benzylidene)-2-(4-nitro-phenyl)-4H-oxazol-5-one;
4-(3,4-Dimethoxy-benzylidene)-2-(2-iodo-phenyl)-4H-oxazol-5-one;
4-(3,4-Dimethoxy-benzylidene)-2-(4-nitro-phenyl)-4H-oxazol-5-one;
4-(2-Hydroxy-naphthalen-1-ylmethylene)-2-phenyl-4H-oxazol-5-one;
4-(4-Nitro-naphthalen-1-ylmethylene)-2-phenyl-4H-oxazol-5-one;
2-(4-Amino-phenyl)-4-(3,4-dimethoxy-benzylidene)-4H-oxazol-5-one;
2-Phenyl-4-(2,3,4-trihydroxy-benzylidene)-4H-oxazol-5-one;
4-(4-Dipentylamino-benzylidene)-2-phenyl-4H-oxazol-5-one;
4-[5-(4-Nitro-phenyl)-furan-2-ylmethylene]-2-p-tolyl-4H-oxazol-5-one;
4-(4-Diethylamino-benzylidene)-2-phenyl-4H-oxazol-5-one;
4-(3-Chloro-benzylidene)-2-(4-fluoro-phenyl)-4H-oxazol-5-one;
4-(3,4-Dichloro-benzylidene)-2-phenyl-4H-oxazol-5-one;
4-(3,4-Dimethyl-benzylidene)-2-phenyl-4H-oxazol-5-one;
4-(4-Fluoro-3-trifluoromethyl-benzylidene)-2-phenyl-4H-oxazol-5-one;
4-(3,4-Dichloro-benzylidene)-2-(4-nitro-phenyl)-4H-oxazol-5-one;
2-Phenyl-4-(2-piperidin-1-yl-cyclohex-1-enylmethylene)-4H-oxazol-5-one;
4-(2,4-Dimethyl-benzylidene)-2-phenyl-4H-oxazol-5-one;
4-(2-Ethoxy-5-methoxymethyl-benzylidene)-2-phenyl-4H-oxazol-5-one;
4-(2-Nitro-benzylidene)-2-phenyl-4H-oxazol-5-one;
4-(3-Nitro-benzylidene)-2-phenyl-4H-oxazol-5-one;
4-(3,4-Dimethoxy-benzylidene)-2-phenyl-4H-oxazol-5-one;
4-(3-Methyl-benzylidene)-2-phenyl-4H-oxazol-5-one;
2-(4-Chloro-phenyl)-4-(1H-indol-3-ylmethylene)-4H-oxazol-5-one;
4-[4-(3,4-Dichloro-benzylidene)-5-oxo-4,5-dihydro-oxazol-2-yl]-benzoic acid;
3-[2-(3,4-Dichloro-phenyl)-5-oxo-oxazol-4-yildenemethyl]-benzoic acid;
(4-Carboxy-phenyl)-{4-[4-(3,4-dichloro-benzylidene)-5-oxo-4,5-dihydro-oxazol-2-yl]-phenyl}-ammonium;
2-[4-(3,4-Dichloro-benzylidene-5-oxo-4,5-dihydro-oxazol-2-yl]-benzoic acid;
2-[2-(3,4-Dichloro-phenyl)-5-oxo-oxazol-4-yildenemethyl]-benzoic acid;
(2-Carboxy-phenyl)-{4-[4-(3,4-dichloro-benzylidene)-5-oxo-4,5-dihydro-oxal-2-yl]-phenyl}-ammonium;
3-[4-(3,4-Dichloro-benzylidene)-5-oxo-4,5-dihydro-oxazol-2-yl]-benzoic acid;
(3-Carboxy-phenyl)-{4-[4-(3,4-dichloro-benzylidene)-5-oxo-4,5-dihydro-oxazol-2-yl]-phenyl}-ammonium;
4-[2-(3,4-Dichloro-phenyl)-5-oxo-oxazol-4-ylidenemethyl]-benzoic acid; and
4-(4-Chloro-3-trifluoromethyl-benzylidene)-2-phenyl-4H-oxazol-5-one.
4-(4-Bromo-benzylidene)-2-phenyl-4H-oxazol-5-one;
2-[2-(2-Bromo-phenyl)-5-oxo-oxazol-4-ylidenemethyl]-benzoic acid methyl ester;
4-[4-(3,4-Dichloro-benzylidene)-5-oxo-4,5-dihydro-oxazol-2-yl]-benzoic acid;
3-[2-(3,4-Dichloro-phenyl)-5-oxo-oxazol-4-ylidenemethyl]-benzoic acid;
(4-Carboxy-phenyl)-{4-[4-(3,4-dichloro-benzylidene)-5-oxo-4,5-dihydro-oxazol-2-yl]-phenyl}-ammonium;
2-[4-(3,4-Dichloro-benzylidene)-5-oxo-4,5-dihydro-oxazol-2-yl]-benzoic acid;
2-[2-(3,4-Dichloro-phenyl)-5-oxo-oxazol-4-ylidenemethyl]-benzoic acid;
(2-Carboxy-phenyl)-{4-[4-(3,4-dichloro-benzylidene)-5-oxo-4,5-dihydro-oxazol-2-yl]-phenyl}-ammonium;
3-[4-(3,4-Dichloro-benzylidene)-5-oxo-4,5-dihydro-oxazol-2-yl]-benzoic acid;
(3-Carboxy-phenyl)-{4-[4-(3,4-dichloro-benzylidene)-5-oxo-4,5-dihydro-oxazol-2-yl]-phenyl}-ammonium;
4-[2-(3,4-Dichloro-phenyl)-5-oxo-oxazol-4-ylidenemethyl]-benzoic acid; and
4-(4-Chloro-3-trifluoromethyl-benzylidene)-2-phenyl-4H-oxazol-5-one.

4. A method of inhibiting the aggregation of amyloid proteins to form amyloid deposits, the method comprising administering to a patient in need of inhibition of the aggregation of amyloid protein an amyloid protein aggregation inhibiting amount of a compound of claim 1.

5. A method of imaging amyloid deposits, the method comprising:
a. introducing into a patient a detectable quantity of a labeled compound according to claim 1;
b. allowing sufficient time for the labeled compound to become associated with amyloid deposits; and
c. detecting the labeled compound associated with the amyloid deposits.

6. The method of claim 5, wherein the patient has or is suspected to have Alzheimer's disease.

7. The method of claim 5, wherein the labeled compound is a radio labeled compound.

8. The method of claim 5, wherein the labeled compound is detected using MRI.

9. A compound of Formula I 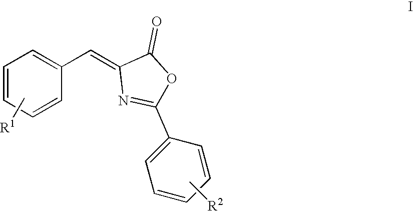
wherein
R¹ is hydrogen, lower alkoxy, hydroxy, aryl, heteroaryl, phenoxy, halogen, hydroxy, cyano, carboxy, alkoxycarbonyl, carbamoyl, sulfamoyl, nitro, trifluoromethyl, amino, mono- or dialkylamino, or lower alkyl or lower alkenyl unsubstituted or substituted with one, two, or three groups independently selected from oxo, halogen, hydroxy, carboxy, carbamoyl, amino, mono- or dialkylamino, or
aryl or heteroaryl optionally substituted independently with up to three groups selected from halogen, lower alkyl, lower alkoxy, hydroxy, carboxy, alkoxycarbonyl, cyano, nitro, trifluoromethyl, amino, mono- or dialkylamino, carbamoyl, carboxyalkyl, alkoxycarbonylalkyl, sulfamoyl, or carbonylamino;
R² is hydrogen, lower alkyl, lower alkoxy, halogen, hydroxy, aryl, heteroaryl, arylalkyl, heteroarylalkyl, arylalkoxy, heteroarylalkoxy, cyano, carboxy, alkoxycarbonyl, carbamoyl, sulfamoyl, nitro, trifluoromethyl, amino, or mono- or dialkylamino,
or the pharmaceutically acceptable salts, esters, amides and prodrugs thereof.

10. A compound according to claim 1, wherein R¹ is hydrogen, halogen, alkoxy, or heteroaryl; and
R² is hydrogen or alkoxy.

11. A compound according to claim 1, wherein the compound is selected from:
4-Benzylidene-2-phenyl-4H-oxazol-5-one;
4-(4-Nitro-benzylidene)-2-phenyl-4H-oxazol-5-one;
2-Acetoxy-5-(5-oxo-2-phenyl-oxazol-4-ylidenemethyl)-benzoic acid;
4-1,3-Benzodioxol-5-ylmethylene-2-phenyl-4H-oxazol-5-one;
4-(2-Methoxy-3,5-dinitro-benzylidene)-2-phenyl-4H-oxazol-5-one;
2-Chloro-N,N-dimethyl-5-[1-(5-oxo-2-phenyl-oxazol-4-ylidene)-ethyl]-benzenesulfonamide;
4-Biphenyl-4-ylmethylene-2-phenyl-4H-oxazol-5-one;
4-(3-Ethoxy-benzylidene)-2-phenyl-4H-oxazol-5-one;
4-(3,5-Dibromo-2-hydroxy-benzylidene)-2-phenyl-4H-oxazol-5-one;
4-(4-Ethoxy-benzylidene)-2-phenyl-4H-oxazol-5-one;
4-(2,6-Dichloro-benzylidene)-2-phenyl-4H-oxazol-5-one;
2,2-Dimethyl-5-[4-(5-oxo-2-phenyl-oxazol-4-ylidenemethyl)-phenoxy]-pentanoic acid;
4-(2-Chloro-cyclohex-1-enylmethylene)-2-phenyl-4H-oxazol-5-one;
2-Phenyl-4-pyridin-3-ylmethylene-4H-oxazol-5-one;
4-(4-Hydroxy-benzylidene)-2-phenyl-4H-oxazol-5-one;
4-(3,4-Dimethoxy-benzylidene)-2-phenyl-4H-oxazol-5-one;
4-Benzylidene-2-(4-nitro-phenyl)-4H-oxazol-5-one;
N-[4-(4-Benzylidene-5-oxo-4,5-dihydro-oxazol-2-yl)-phenyl]-acetamide;
4-Benzylidene-2-(4-chloro-phenyl)-4H-oxazol-5-one;
4-Benzylidene-2-p-tolyl-4H-oxazol-5-one;
4-Benzylidene-2-(4-methoxy-phenyl)-4H-oxazol-5-one;
4-Benzo[1,3]dioxol-5-ylmethylene-2-phenyl-4H-oxazol-5-one;
2-(4-Chloro-3-nitro-phenyl)-4-(4-diethylamino-benzylidene)-4H-oxazol-5-one;
4-(4-Dimethylamino-naphthalen-1-ylmethylene)-2-phenyl-4H-oxazol-5-one;
4-(3,5-Dichloro-2-hydroxy-benzylidene)-2-phenyl-4H-oxazol-5-one;
4-(2,3-Dihydroxy-benzylidene)-2-phenyl-4H-oxazol-5-one;
4-(4-Phenoxy-benzylidene)-2-phenyl-4H-oxazol-5-one;
4-(4-Dimethylamino-benzylidene)-2-phenyl-4H-oxazol-5-one;
4-(4-Dimethylamino-benzylidene)-2-phenyl-4H-oxazol-5-one;
4-Naphthalen-1-ylmethylene-2-phenyl-4H-oxazol-5-one; 4-Naphthalen-2-ylmethylene-2-phenyl-4H-oxazol-5-one;
4-(4-Dipropylamino-benzylidene)-2-phenyl-4H-oxazol-5-one;
4-(4-Diethylamino-benzylidene)-2-p-tolyl-4H-oxazol-5-one;
4-(4-Diethylamino-benzylidene)-2-(3,5-dinitro-phenyl)-4H-oxazol-5-one;
4-(2,4-Dichloro-benzylidene)-2-phenyl-4H-oxazol-5-one;
2-(4-Chloro-3-nitro-phenyl)-4-pyridin-4-ylmethylene-4H-oxazol-5-one;
2-(4-Chloro-phenyl)-4-pyridin-2-ylmethylene-4H-oxazol-5-one;
4-(2-Hydroxy-5-nitro-benzylidene)-2-phenyl-4H-oxazol-5-one;
2-(4-Chloro-3-nitro-phenyl)-4-(4-fluoro-benzylidene)-4H-oxazol-5-one;
4-(4-Dimethylamino-benzylidene)-2-(3-nitro-phenyl)-4H-oxazol-5-one;
4-(4-Chloro-benzylidene)-2-(3-nitro-phenyl)-4H-oxazol-5-one;
4-(4-Methoxy-benzylidene)-2-(3-nitro-phenyl)-4H-oxazol-5-one;
4-(4-Diethylamino-benzylidene)-2-(3-nitro-phenyl)-4H-oxazol-5-one;
4-(4-Methyl-benzylidene)-2-(3-nitro-phenyl)-4H-oxazol-5-one;
2-(4-Bromo-phenyl)-4-(4-diethylamino-benzylidene)-4H-oxazol-5-one;
2-(2-Chloro-phenyl)-4-(4-diethylamino-benzylidene)-4H-oxazol-5-one;
2-(3-Nitro-phenyl)-4-pyridin-4-ylmethylene-4H-oxazol-5-one;
4-(2,3-Dimethoxy-benzylidene)-2-(2-iodo-phenyl)-4H-oxazol-5-one;
4-(2-Methoxy-benzylidene)-2-phenyl-4H-oxazol-5-one;
4-(3-Methoxy-benzylidene)-2-phenyl-4H-oxazol-5-one;
4-(4-Methoxy-benzylidene)-2-phenyl-4H-oxazol-5-one;
4-(2,3-Dimethoxy-benzylidene)-2-phenyl-4H-oxazol-5-one;
4-(3,4-Dimethoxy-benzylidene)-2-(2-hydroxy-phenyl)-4H-oxazol-5-one;
4-(2,3-Dimethoxy-benzylidene)-2-(4-nitro-phenyl)-4H-oxazol-5-one;
4-(3,4-Dimethoxy-benzylidene)-2-(2-iodo-phenyl)-4H-oxazol-5-one;
4-(3,4-Dimethoxy-benzylidene)-2-(4-nitro-phenyl)-4H-oxazol-5-one;
4-(2-Hydroxy-naphthalen-1-ylmethylene)-2-phenyl-4H-oxazol-5-one;
4-(4-Nitro-naphthalen-1-ylmethylene)-2-phenyl-4H-oxazol-5-one;
2-(4-Amino-phenyl)-4-(3,4-dimethoxy-benzylidene)-4H-oxazol-5-one;
2-Phenyl-4-(2,3,4-trihydroxy-benzylidene)-4H-oxazol-5-one;
4-(4-Dipentylamino-benzylidene)-2-phenyl-4H-oxazol-5-one;
4-[5-(4-Nitro-phenyl)-furan-2-ylmethylene]-2-p-tolyl-4H-oxazol-5-one;
4-(4-Diethylamino-benzylidene)-2-phenyl-4H-oxazol-5-one;
4-(3-Chloro-benzylidene)-2-(4-fluoro-phenyl)-4H-oxazol-5-one;
4-(3,4-Dichloro-benzylidene)-2-phenyl-4H-oxazol-5-one;
4-(3,4-Dimethyl-benzylidene)-2-phenyl-4H-oxazol-5-one;
4-(4-Fluoro-3-trifluoromethyl-benzylidene)-2-phenyl-4H-oxazol-5-one;
4-(3,4-Dichloro-benzylidene)-2-(4-nitro-phenyl)-4H-oxazol-5-one;
2-Phenyl-4-(2-piperidin-1-yl-cyclohex-1-enylmethylene)-4H-oxazol-5-one;
4-(2,4-Dimethyl-benzylidene)-2-phenyl-4H-oxazol-5-one;
4-(2-Ethoxy-5-methoxymethyl-benzylidene)-2-phenyl-4H-oxazol-5-one;
4-(2-Nitro-benzylidene)-2-phenyl-4H-oxazol-5-one;
4-(3-Nitro-benzylidene)-2-phenyl-4H-oxazol-5-one;
4-(3,4-Dimethoxy-benzylidene)-2-phenyl-4H-oxazol-5-one;
4-(3-Methyl-benzylidene)-2-phenyl-4H-oxazol-5-one;
2-(4-Chloro-phenyl)-4-(1H-indol-3-ylmethylene)-4H-oxazol-5-one;
4-[4-(3,4-Dichloro-benzylidene)-5-oxo-4,5-dihydro-oxazol-2-yl]-benzoic acid;
3-[2-(3,4-Dichloro-phenyl)-5-oxo-oxazol-4-yildenemethyl]-benzoic acid;
(4-Carboxy-phenyl)-{4-[4-(3,4-dichloro-benzylidene)-5-oxo-4,5-dihydro-oxazol-2-yl]-phenyl}-ammonium;
2-[4-(3,4-Dichloro-benzylidene-5-oxo-4,5-dihydro-oxazol-2-yl]-benzoic acid;
2-[2-(3,4-Dichloro-phenyl)-5-oxo-oxazol-4-yildenemethyl]-benzoic acid;
(2-Carboxy-phenyl)-{4-[4-(3,4-dichloro-benzylidene)-5-oxo-4,5-dihydro-oxal-2-yl]-phenyl}-ammonium;
3-[4-(3,4-Dichloro-benzylidene)-5-oxo-4,5-dihydro-oxazol-2-yl]-benzoic acid;
(3-Carboxy-phenyl)-{4-[4-(3,4-dichloro-benzylidene)-5-oxo-4,5-dihydro-oxazol-2-yl]-phenyl}-ammonium;
4-[2-(3,4-Dichloro-phenyl)-5-oxo-oxazol-4-ylidenemethyl]-benzoic acid; and
4-(4-Chloro-3-trifluoromethyl-benzylidene)-2-phenyl-4H-oxazol-5-one.
4-(4-Bromo-benzylidene)-2-phenyl-4H-oxazol-5-one;
2-[2-(2-Bromo-phenyl)-5-oxo-oxazol-4-ylidenemethyl]-benzoic acid methyl ester;
4-[4-(3,4-Dichloro-benzylidene)-5-oxo-4,5-dihydro-oxazol-2-yl]-benzoic acid;
3-[2-(3,4-Dichloro-phenyl)-5-oxo-oxazol-4-ylidenemethyl]-benzoic acid;
(4-Carboxy-phenyl)-{4-[4-(3,4-dichloro-benzylidene)-5-oxo-4,5-dihydro-oxazol-2-yl]-phenyl}-ammonium;
2-[4-(3,4-Dichloro-benzylidene)-5-oxo-4,5-dihydro-oxazol-2-yl]-benzoic acid;
2-[2-(3,4-Dichloro-phenyl)-5-oxo-oxazol-4-ylidenemethyl]-benzoic acid;
(2-Carboxy-phenyl)-{4-[4-(3,4-dichloro-benzylidene)-5-oxo-4,5-dihydro-oxazol-2-yl]-phenyl}-ammonium;
3-[4-(3,4-Dichloro-benzylidene)-5-oxo-4,5-dihydro-oxazol-2-yl]-benzoic acid;
(3-Carboxy-phenyl)-{4-[4-(3,4-dichloro-benzylidene)-5-oxo-4,5-dihydro-oxazol-2-yl]-phenyl}-ammonium;
4-[2-(3,4-Dichloro-phenyl)-5-oxo-oxazol-4-ylidenemethyl]-benzoic acid; and
4-(4-Chloro-3-trifluoromethyl-benzylidene)-2-phenyl-4H-oxazol-5-one.

12. A composition comprising a compound of claim 1 and a pharmaceutically acceptable carrier.
